(19) United States
(12) Patent Application Publication
(10) Pub. No.: US 2023/0073915 A1
(43) Pub. Date: Mar. 9, 2023

(54) ULTRASONIC TRANSDUCER AND ULTRASONIC TREATMENT INSTRUMENT

(71) Applicant: OLYMPUS MEDICAL SYSTEMS CORP., Tokyo (JP)

(72) Inventors: Yusuke INUI, Kawasaki-shi, Kanagawa (JP); Yukihiko SHIMAMURA, Yoshikawa-shi, Saitama (JP); Keisuke NAGAO, Hachioji-shi, Tokyo (JP)

(73) Assignee: OLYMPUS MEDICAL SYSTEMS CORP., Tokyo (JP)

(21) Appl. No.: 17/900,984

(22) Filed: Sep. 1, 2022

Related U.S. Application Data
(60) Provisional application No. 63/242,069, filed on Sep. 9, 2021.

(51) Int. Cl. A61B 18/14; B06B 1/06

(52) U.S. Cl. CPC A61B 18/1445; B06B 1/0611; A61B 2018/00994

(57) ABSTRACT

The ultrasonic transducer has a cylindrical shape having a first end surface and a second end surface, and includes a piezoelectric material that generates ultrasonic vibration for treating biological tissue with ultrasonic energy, a first electrode that is disposed in contact with the first end surface and to which an ultrasonic driving voltage is applied for generating ultrasonic vibration to the piezoelectric material, a second electrode that is disposed in contact with the second end surface and to which a reference voltage is applied for generating ultrasonic vibration to the piezoelectric material, an insulating plate that opposes the second end surface with the second electrode interposed therebetween, a third electrode that opposes the second electrode with the insulating plate and to which high-frequency power for treating biological tissue is supplied with high-frequency energy, and a short-circuit prevention unit that prevents short-circuiting between the first and third electrodes.

### RELATED APPLICATION DATA

This application is based on and claims priority under 37 U.S.C. § 119 to U.S. Provisional Application No. 63/242,069 filed on Sep. 9, 2021, the entire contents of which is incorporated herein by reference.

### FIELD OF THE INVENTION

The present invention relates to an ultrasonic transducer and an ultrasonic treatment instrument.

### DESCRIPTION OF THE RELATED ART

Conventionally, there has been known an ultrasonic treatment instrument which treats a site of interest by imparting ultrasonic energy and high frequency energy to a site (hereinafter, described as a target site) to be treated in a biological tissue (see, for example, Patent Document 1). The ultrasonic treatment instrument described in Patent Document 1 includes an ultrasonic transducer for generating ultrasonic vibration for treating a target site by ultrasonic energy. The ultrasonic transducer comprises a piezoelectric material (or piezoelectric element), a first electrode, a second electrode, an insulating plate, and a third electrode as described below. Piezoelectric elements have a cylindrical shape having first and second end faces formed front and back to each other, to generate ultrasonic vibration. The first electrode is disposed in contact with the first end face and applies an ultrasonic drive voltage for generating ultrasonic vibration to the piezoelectric element. The second electrode is disposed in contact with the second end face and applies a reference voltage for generating ultrasonic vibration to the piezoelectric element. An insulating plate is composed of a material having an electrically insulating property and is disposed opposed to the second end face across the second electrode and in contact with the second electrode. The third electrode is opposed to the second electrode with the insulation plate interposed therebetween, and supplies high frequency power for treating the target site by high frequency energy.

Prior art documents—Patent Document 1: International Publication No. WO 2020/152809.

### SUMMARY OF THE INVENTION

### Problem to be Solved by the Invention

In the ultrasonic transducer described in Patent Document 1, depending on the design, it is not possible to sufficiently secure the minimum distance between conductive parts along the surface of the insulator (also called creepage distance) and the space distance between the first electrode and the third electrode, and it may not be possible to sufficiently prevent a short circuit between the first electrode and the third electrode. Therefore, a technique capable of preventing a short circuit between the first electrode and the third electrode is desired.

In view of the above, it is an object of the present invention to provide an ultrasonic transducer and an ultrasonic treatment instrument capable of more reliably preventing a short circuit between a first electrode and a third electrode.

### Means for Solving the Problem

In order to solve the above problems and achieve the purpose, the ultrasonic transducer according to the present invention is a piezoelectric material having a cylindrical shape, the piezoelectric material including a first surface and a second surface opposing the first surface, the piezoelectric material configured to generate an ultrasonic vibration for treating living tissue by an ultrasonic energy. The first electrode contacts the first surface of the piezoelectric material and is configured to apply an ultrasonic driving voltage to the piezoelectric material for generating the ultrasonic vibration, and the second electrode contacts the second surface of the piezoelectric material and is configured to apply a reference voltage to the piezoelectric material for generating the ultrasonic vibration. An insulation plate having an electrical insulating property is arranged on the second surface side of the piezoelectric material with the second electrode interposed therebetween and the first side of the insulation plate contacting the second electrode. A third electrode is arranged on the second side of the insulation plate and is configured to apply a high-frequency electric power for treating living tissue by a high-frequency energy. A short-circuit prevention portion configured to prevent short-circuit is located between the first electrode and the third electrode.

An ultrasonic treatment device according to the present invention includes an end effector for treating biological tissue by imparting ultrasonic energy and high frequency energy to the biological tissue, and an ultrasonic transducer for generating ultrasonic vibration. The ultrasonic transducer according to the present invention is a piezoelectric material having a cylindrical shape, the piezoelectric material including a first surface and a second surface opposing the first surface, the piezoelectric material configured to generate an ultrasonic vibration for treating living tissue by an ultrasonic energy. The first electrode contacts the first surface of the piezoelectric material and is configured to apply an ultrasonic driving voltage to the piezoelectric material for generating the ultrasonic vibration, and the second electrode contacts the second surface of the piezoelectric material and is configured to apply a reference voltage to the piezoelectric material for generating the ultrasonic vibration. An insulation plate having an electrical insulating property is arranged on the second surface side of the piezoelectric material with the second electrode interposed therebetween and the first side of the insulation plate contacting the second electrode. A third electrode is arranged on the second side of the insulation plate and is configured to apply a high-frequency electric power for treating living tissue by a high-frequency energy. A short-circuit prevention portion configured to prevent short-circuit is located between the first electrode and the third electrode.

### Effect of the Invention

According to the ultrasonic transducer and the ultrasonic treatment instrument according to the present invention, it is possible to more reliably prevent a short circuit between the first electrode and the third electrode.

### BRIEF DESCRIPTION OF THE DRAWINGS

FIG. 1 is a diagram illustrating a treatment system according to the first embodiment.

FIG. 2 is a diagram illustrating a configuration of an ultrasonic treatment instrument.

FIG. 3 is a diagram illustrating a configuration of an ultrasonic treatment instrument.

FIG. 4 is a diagram illustrating a configuration of an ultrasonic treatment instrument.

FIG. 5 is a diagram for explaining the configuration of the short-circuit prevention portion.

FIG. 6 is a diagram for explaining the configuration of the short-circuit prevention portion.

FIG. 7 is a diagram illustrating a modification 1-1 of the first embodiment.

FIG. 8 is a diagram illustrating a modification 1-2 of the first embodiment.

FIG. 9 is a diagram illustrating a modification 1-2 of the first embodiment.

FIG. 10 is a diagram illustrating a modification 1-3 of the first embodiment.

FIG. 11 is a diagram illustrating a modification 1-4 of the first embodiment.

FIG. 12 is a diagram illustrating a modification 1-5 of the first embodiment.

FIG. 13 is a diagram illustrating a modification 1-6 of the first embodiment.

FIG. 14 is a diagram illustrating a modification 1-7 of the first embodiment.

FIG. 15 is a diagram illustrating a modification 1-8 of the first embodiment.

FIG. 16 is a diagram illustrating a modification 1-9 of the first embodiment.

FIG. 17 is a diagram illustrating a modification 1-10 of the first embodiment.

FIG. 18 is a diagram illustrating a modification 1-11 of the first embodiment.

FIG. 19 is a diagram illustrating a modification 1-12 of the first embodiment.

FIG. 20 is a diagram illustrating a modification 1-13 of the first embodiment.

FIG. 21 is a diagram illustrating a configuration of a short circuit prevention portion according to the second embodiment.

FIG. 22 is a diagram illustrating a modification 2-1 of the second embodiment.

FIG. 23 is a diagram illustrating a modification 2-2 of the second embodiment.

FIG. 24 is a diagram illustrating a modification 2-3 of the second embodiment.

FIG. 25 is a diagram illustrating a modification 2-4 of the second embodiment.

FIG. 26 is a diagram illustrating a modification 2-5 of the second embodiment.

FIG. 27 is a diagram illustrating a modification 2-6 of the second embodiment.

FIG. 28 is a diagram illustrating a modification 2-7 of the second embodiment.

FIG. 29 is a diagram illustrating a modification 2-8 of the second embodiment.

FIG. 30 is a diagram illustrating a modification 2-9 of the second embodiment.

FIG. 31 is a diagram illustrating a modification 2-10 of the second embodiment.

FIG. 32 is a diagram illustrating a modification 2-11 of the second embodiment.

FIG. 33 is a diagram illustrating a modification 2-12 of the second embodiment.

FIG. 34 is a diagram illustrating a modification 2-13 of the second embodiment.

### DETAILED DESCRIPTION OF THE PREFERRED EMBODIMENT(S)

### Modes for Carrying Out the Invention

Hereinafter, embodiments for carrying out the present invention (hereinafter, embodiments) will be described with reference to the accompanying drawings. Note that the present invention is not limited by the embodiments described below. In addition, in the description of the drawings, the same parts are denoted by the same reference numerals.

Schematic Configuration of the Treatment System

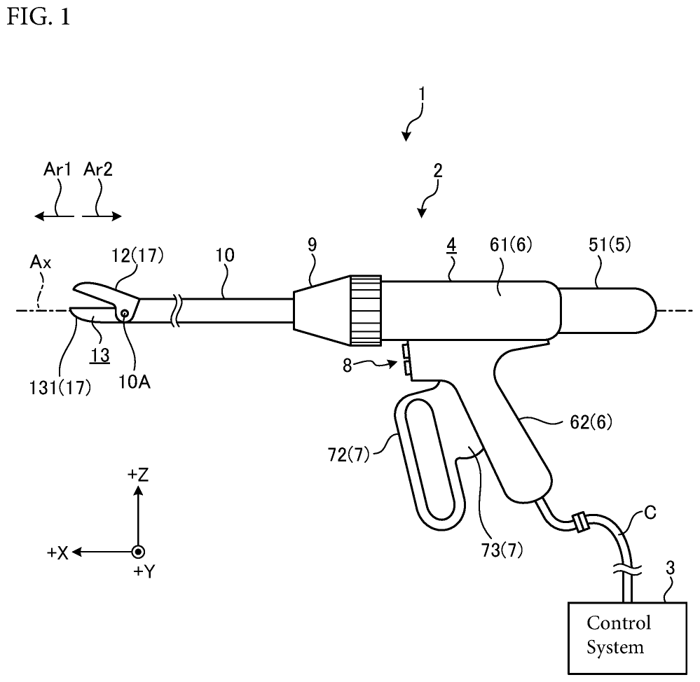

FIG. 1 is a diagram illustrating a treatment system 1 according to the first embodiment. This treatment system 1 comprises an ultrasonic treatment instrument 2 and a control system 3 as shown in FIG. 1. The treatment system 1 imparts treatment energy to a site to be treated in a biological tissue (hereinafter, described as a target site). Note that the procedure energy in the first embodiment is ultrasonic energy and high frequency energy, but embodiments can include other procedure energies. Further, a process that can be performed by the treatment system 1 according to the first embodiment is coagulation (sealing) of a target site, incision of a target site, but other treatment operations can be conducted with the treatment system. In addition, treatment operations, such as coagulation and incision, may be performed simultaneously.

Composition of the Ultrasound Treatment Device

In the following, in describing the configuration of the ultrasonic treatment instrument 2, the X-axis, Y-axis, and Z-axis are mutually orthogonal, as shown by the XYZ coordinate axis in FIG. 1. The X-axis is an axis parallel to the central axis Ax of the shaft 10 (FIG. 1), the Y-axis is an axis perpendicular to the plane of the paper, and the Z-axis is an axis along the vertical direction of FIG. 1. In addition, in the following, one side along the central axis Ax (+X-axis side) is described as a distal end side Ar1, and the other side (−X-axis side) is described as a proximal end side Ar2.

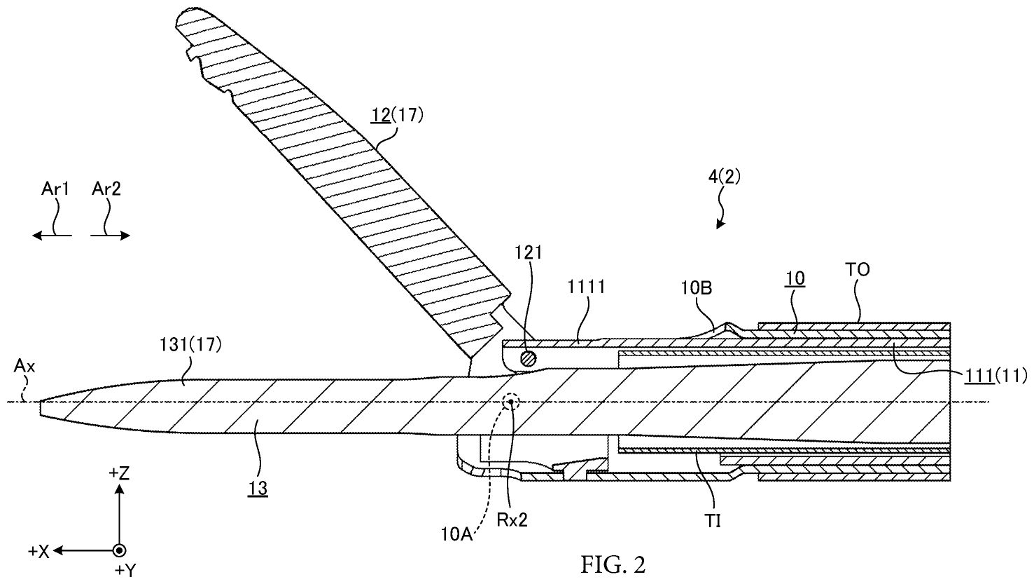

FIGS. 2 to 4 are views illustrating a configuration of an ultrasonic treatment instrument 2. Specifically, FIGS. 2 to 4 are cross sectional views from the +Y-axis side and cutting the ultrasonic treatment instrument 2 in the XZ plane including the central axis Ax. The portions of the ultrasonic treatment instrument 2 in the views in FIGS. 2 to 4 are illustrated in order from the distal end side Ar1 to the proximal end side Ar2.

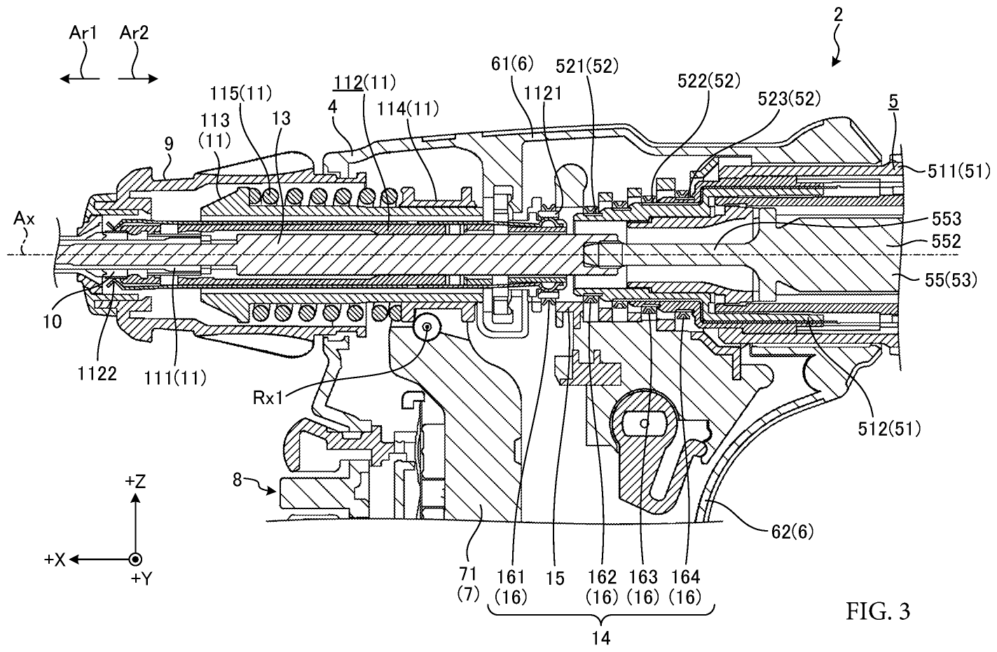

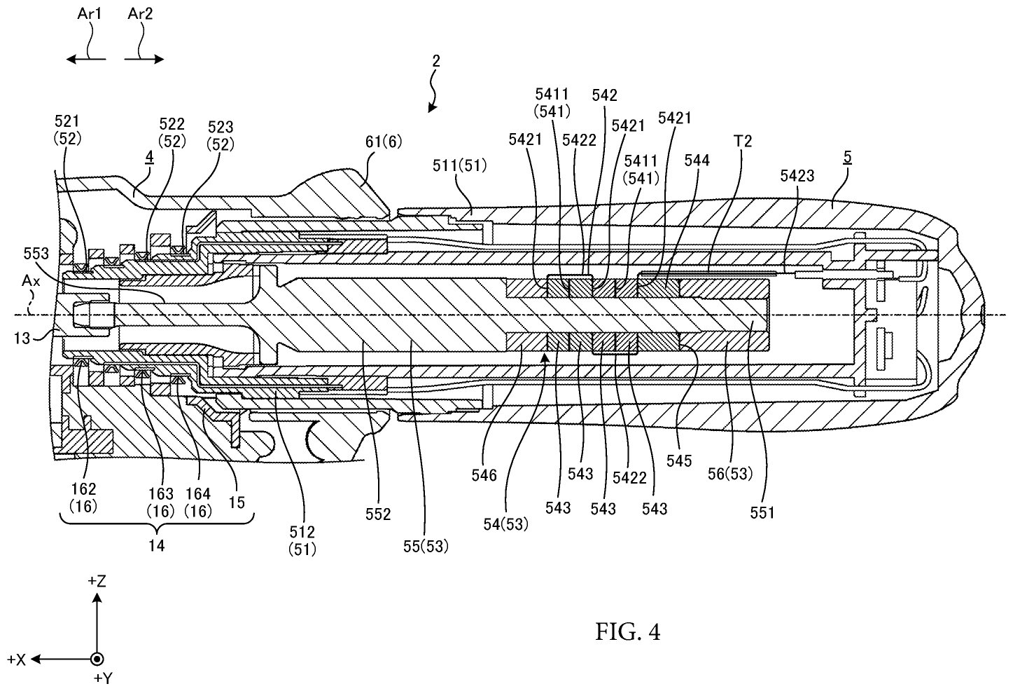

The ultrasonic treatment instrument 2 is an ultrasonic treatment instrument for treating the target site by imparting ultrasonic energy and high frequency energy to the target site. The ultrasonic treatment instrument 2 comprises a handpiece 4 and an ultrasonic transducer 5, as shown in FIGS. 1 to 4. As shown in FIGS. 1 to 4, the handpiece 4 includes a housing 6 (FIGS. 1, 3, and 4), a movable handle 7 (FIGS. 1 and 3), a switch 8 (FIGS. 1 and 3), a rotary knob 9 (FIGS. 1 and 3), a shaft 10 (FIGS. 1 to 3), an opening and closing mechanism 11 (FIGS. 2 and 3), a jaw 12 (FIGS. 1 and 2), a vibration transmission member 13 (FIGS. 1 to 3), and a terminal unit 14 (FIGS. 3 and 4).

The housing 6 supports the entire ultrasonic treatment instrument 2. The housing 6, as shown in FIG. 1, 3, or 4, comprises a substantially cylindrical case body 61 which is coaxial with the central axis Ax, and a fixed handle 62 which extends from the case body 61 to the −Z axis side and is held by an operator, such as a medical professional.

The movable handle 7 accepts a user operation (a closing operation and an opening operation) by an operator. The movable handle 7, as shown in FIG. 1 and FIG. 3, includes a handle base 71 (FIG. 3), an operation unit 72 (FIG. 1), and a connecting portion 73 (FIG. 1).

Handle base 71 is located within housing 6. The +Z-axis-side portion of the handle base portion 71, with respect to the housing 6, is rotatably supported about a first rotational axis Rx1 which is parallel to the Y-axis (FIG. 3). Further, the end of the +Z-axis side of the end portion of the handle base 71 extends toward the +Z-axis side, is bifurcated into branches, and functions to engage the slider 114 that constitutes the opening and closing mechanism 11 (not shown).

As shown in FIG. 1, the operation unit 72 is a portion located outside the housing 6 and is configured to receive the closing operation and the opening operation by an operator, respectively. Also as shown in FIG. 1, connecting portion 73 is a portion for connecting the handle base 71 and the operation unit 72 and is disposed across the inside and outside of the housing 6. In operation, when receiving a closing operation by an operator, the movable handle 7 rotates counterclockwise around the first rotational axis Rx1 (see FIG. 3). In other words, the operation unit 72 moves in a direction toward the fixed handle 62. Also in operation, when receiving an opening operation by an operator, the movable handle 7 rotates clockwise around the first rotational axis Rx1 (see FIG. 3). In other words, the operation unit 72 moves in a direction away from the fixed handle 62.

As shown in FIG. 1 and FIG. 3, switch 8 is provided such that a portion is exposed to the outside from the side surface of the distal end-side Ar1 in the handle body 62. The switch 8 is operable by an operator to initiate a treatment operation that imparts treatment energy to the site of interest.

Rotary knob 9 has a substantially cylindrical shape or conical shape that is coaxial with the central axis Ax, as shown in FIG. 1 and FIG. 3, and is provided on the distal end side Ar1 of the case body 61. Then, the rotary knob 9 accepts a rotation control, which is a user operation by an operator. By the rotation control, the rotary knob 9 rotates about the central axis Ax with respect to the case body 61. Further, in addition to the rotary knob 9 being rotatable, the jaw 12 and the vibration transmission member 13 also can be rotatable about the central axis Ax.

Shaft 10 is a cylindrical pipe made of a conductive material such as metal. Additionally, the outer peripheral surface of the shaft 10 is covered by an electrically insulating outer tube TO (FIG. 2). Further, the end portion of the distal end side Ar1 of the shaft 10 extends along the Y-axis and supports pin 10A that rotatably supports jaw 12 to rotate around the second rotational axis Rx2 (FIG. 1, FIG. 2). Furthermore, at the end of the distal end side Ar1 of the shaft 10 and to the +Z-axis side of the shaft 10, notches 102 are formed that extend toward the proximal end side Ar2 and open toward the distal end side Ar1 (FIG. 2).

The jaw 12 has an opening and closing mechanism 11 that causes elements of the jaw 12 to rotate around the second rotational axis Rx2 in response to the opening operation and closing operation affected by manipulation of the movable handle 7 by an operator. Then, by the opening and closing mechanism 11, the jaw 12 opens and closes with respect to the end portion 131 (hereinafter, referred to as the treatment portion 131 (FIG. 2)) on the distal end side Ar1 of the vibration transmission member 13 and grasps the target site between the jaw 12 and the treatment portion 131.

As shown in FIG. 2 and FIG. 3, the opening and closing mechanism 11 includes an inner pipe 111, a holding portion 112 (FIG. 3), a slider receiver 113 (FIG. 3), a slider 114 (FIG. 3), and a biasing member 115 (FIG. 3). The inner pipe 111 is a cylindrical pipe having a smaller diameter than the shaft 10. Further, the inner pipe 111 is coaxial with the shaft 10 and is inserted into the interior of the shaft 10. In the inner pipe 111, the +Z-axis side of the end portion of the distal end side Ar1 has an arm portion 1111 that extends toward the distal end side Ar1 (FIG. 2). The arm portion 1111 is attached to the jaw 12 by a second pin 121 which is inserted parallel to the second rotary shaft Rx2 (i.e., is oriented parallel to the first pin 10A).

Holding portion 112 is constituted by a material having an electrical insulating property such as a resin, and has a substantially cylindrical shape. The holding portion 112 is inserted into the rotary knob 9 and the case body 61 in a state straddling the rotary knob 9 and the case body 61 as shown in FIG. 3. The holding portion 112 holds the vibration transmission member 13 inserted therein. The distal end side Ar1 of the holding portion 112 is mechanically connected to the rotary knob 9 and the shaft 10. That is, in accordance with the rotation control to the rotary knob 9 by an operator, the holding portion 112, the shaft 10, the jaw 12, and the vibration transmission member 13 rotate about the central axis Ax together with the rotary knob 9.

As shown in FIG. 3, the holding portion 112 includes the HF return electrode terminal 1121 and the electrical path 1122.

HF return electrode terminal 1121 is composed of a conductive material and has a ring shape that extends over the entire circumference in the circumferential direction around the central axis Ax. Further, HF return electrode terminal 1121 is attached to the outer peripheral surface of the proximal end side Ar2 in the holding portion 112. HF return electrode terminal 1121 is electrically connected to the HF return electrode terminal 161 provided in the terminal unit 14 (FIG. 3). Since the HF return electrode terminal 1121 has a ring shape as described above, in response to the rotation control to the rotary knob 9 by an operator, even when rotated about the central axis Ax, the HF return electrode terminal 161 is always electrically connected to the HF return electrode terminal 161.

Electrical path 1122 is made of a conductive material and extends from the end of the proximal end side Ar2 to the end of the distal end side Ar1 at the outer peripheral surface of the holding portion 112. As shown in FIG. 3, the end of the proximal end side Ar2 of the electric path 1122 is electrically connected to the HF return electrode terminal 1121 and the end of the distal end side Ar1 of the electric path 1122 is electrically connected to the shaft 10.

Slider receiver 113 is composed of a material having an electrical insulation, such as resin, and has a substantially cylindrical shape. The slider receiver 113, while the holding portion 112 is inserted therein, is movably disposed along the central axis Ax with respect to the holding portion 112. Here, the end of the distal end side Ar1 of the slider receiver 113 is connected to the end of the proximal end side Ar2 of the inner pipe 111 such that movement along the central axis Ax is possible with respect to the holding section 112, but rotation around the central axis Ax is restricted. That is, in response to the rotation control to the rotary knob 9 by an operator, the slider receiver 113 and the inner pipe 111 rotate about the central axis Ax together with the rotary knob 9.

The slider 114 has a substantially cylindrical shape and is disposed to be movable along a central axis Ax with respect to the slider receiver 113. The slider 114 is engage with a pair of engaging portions (not shown) in the movable handle 7.

The opening/closing mechanism 11 operates as described below in response to an operation on the movable handle 7 by an operator.

In response to closing operation to the movable handle 7 by an operator, slider 114 is pushed toward the distal end side Ar1 along the central axis Ax by a pair of engaging portions in the movable handle 7 (not shown). Further, the slider receiver 113 is subjected to a pressing force toward the distal end side Ar1 resulting from the slider 114 movement and is passed through a biasing member 115 disposed between the slider 114 and the slider receiver 113, causing the slider receiver 113 to move towards the distal end-side Ar1 along the central axis Ax. In conjunction with the movement of the slider receiver 113, the inner pipe 111 moves toward the distal end side Ar1 along the central axis Ax. In conjunction with the movement of the inner pipe 111, the arm portion 1111 pushes the pin 121 toward the distal end side Ar1. Then, the jaw 12 rotates counterclockwise in FIG. 2 about the second rotational axis Rx2. At this time, since the pin 121 also moves while maintaining a constant distance about the second rotational axis Rx2, the arm portion 1111 moves toward the distal end side Ar1 while deforming the +Z-axis side notch 10B. In other words, the jaw 12 moves in a direction (closing direction) proximate to the treatment portion 131. Further, in accordance with the opening operation applied to the movable handle 7 by an operator, the jaw 12 in FIG. 2 rotates clockwise around the second rotational axis Rx2. In other words, the jaw 12 moves in a direction (opening direction) spaced apart from the treatment portion 131. As described above, in response to an operation on the movable handle 7 by an operator, the jaw 12 opens and closes with respect to the treatment portion 131 and grasps the target site between the jaw 12 and the treatment portion 131.

The biasing member 115 can be, for example, a coil spring. The biasing member 115 acts on the slider 114 to bias the movable handle 7 and contributes a gripping force for grasping the target site between the jaw 12 and the treatment portion 131. More specifically, the biasing member 115 is used to make the gripping force constant.

Jaw 12 is at least partially composed of a conductive material. The jaw 12 is electrically connected to the HF return electrode terminal 1121 by passing through the shaft 10 and the electrical path 1122.

Vibration transmission member 13 is composed of a conductive material and has an elongated shape extending linearly along the central axis Ax. Further and as shown in FIG. 2, the treatment portion 131 of the vibration transmission member 13 is located in the inner pipe 111 and projects to the outside. The proximal end side Ar2 of the vibration transmission member 13 mechanically connects to the ultrasonic transducer 5, as shown in FIG. 3. In response to the rotation control to the rotary knob 9 by an operator, the ultrasonic transducer 5 rotates about the central axis Ax together with the vibration transmission member 13. The vibration transmission member 13 transmits the ultrasonic vibration generated by the ultrasonic transducer 5 from the proximal end side Ar2 to the treatment portion 131. In the first embodiment, the ultrasonic vibration is a longitudinal vibration vibrating in a direction along the central axis Ax. In order to ensure electrical insulation between the shaft 10 and the inner pipe 111, the outer peripheral surface of the vibration transmission member 13 is covered by an electrically insulating inner tube TI (FIG. 2).

Terminal unit 14 is a portion for electrically connecting the electrical cable C (FIG. 1) and the ultrasonic transducer 5 and the HF return electrode terminal 1121. As shown in FIG. 3 and FIG. 4, the terminal unit 14 includes a terminal holding member 15 and a handpiece side terminal 16. Terminal holding member 15 is composed of a material having an electrically insulating property and, as shown in FIG. 3 or FIG. 4, has a cylindrical body having a generally truncated cone shape with a central axis that is coaxial with the central axis Ax and the larger diameter side (of the truncated cone shape) is disposed inside the case body 61 in a position facing the proximal end side Ar2.

As shown in FIG. 3 and FIG. 4, handpiece side terminal 16 includes an HF return electrode terminal 161, an HF active electrode terminal 162, an ultrasound (US) return electrode terminal 163, and an U.S. active electrode terminal 164. Each of these terminals 161-164 is made of a conductive material.

HF return electrode terminal 161 is located on the most distal end side Ar1 of terminals 161-164. A portion of the HF return electrode terminal 161 is attached to the terminal holding member 15 and is also exposed to the inside of the terminal holding member 15. The HF return electrode terminal 161 is configured to be electrically connected to the HF return electrode cable via the electrical cable C (not shown).

HF active electrode terminal 162 is located on the proximal end side Ar2 of and separated from the HF return electrode terminal 161. A portion of the HF active electrode terminal 162 is attached to the terminal holding member 15 and is also exposed to the inside of the terminal holding member 15. The HF active electrode terminal 162 is configured to be electrically connected to the HF active electrode cable via the electrical cable C (not shown).

U.S. return electrode terminal 163 is located on the proximal end side Ar2 of and separated from the HF active electrode terminal 162. A portion of the U.S. return electrode terminal 163 is attached to the terminal holding member 15 and is also exposed to the inside of the terminal holding member 15. The U.S. return electrode terminal 163 is configured to be electrically connected to the U.S. return electrode cable via the electrical cable C (not shown).

U.S. active electrode terminal 164 is positioned on the most proximal end side Ar2 of the terminals 161-164. A portion of the U.S. active electrode terminal 164 is attached to the terminal holding member 15 and is also exposed to the inside of the terminal holding member 15. The U.S. active electrode terminal 164 is configured to be electrically connected to the U.S. active electrode cable via the electrical cable C (not shown).

As shown in FIG. 4, the ultrasonic transducer 5 includes a TD (transducer) case 51, a TD side terminal 52, and an ultrasonic vibrator 53. TD case 51 supports the TD side terminal 52 and the ultrasonic vibrator 53 and is detachably connected to the case body 61.

As shown in FIG. 4, the TD case 51 includes a TD case body 511 and a terminal holding portion 512. As shown in FIG. 2, TD case body 511 has a bottomed cylindrical shape and an opening portion in a position facing the distal end side Ar1. The opening portion of the TD case body 511 is connected to the case body 61. When the ultrasonic transducer 5 is inserted into the case body 61 (connection), the central axis of the ultrasonic transducer 5 is oriented matching the central axis Ax. As shown in FIG. 4, terminal holding portion 512 has a cylindrical body having a generally truncated cone shape with a central axis that is coaxial with the central axis of the ultrasonic transducer 5 and the large diameter side (of the truncated cone shape) is fitted to the opening in the TD case body 511 in a position facing the proximal end side Ar2.

As shown in FIG. 3 and FIG. 4, TD side terminal 52 is provided on the outer peripheral surface of the terminal holding portion 512. The TD-side terminal 52 includes an HF active electrode terminal 521, a U.S. return electrode terminal 522, and a US active electrode terminal 523. Each of these terminals 521-523 is made of a conductive material.

The outer peripheral surface of the terminal holding portion 512 includes HF active electrode terminal 521, which is disposed on the most distal end side Ar1 of the terminals 521 to 523. HF active electrode terminal 521 is provided over the entire circumference of the terminal holding portion 512 in the circumferential direction around the central axis Ax. Then, by connecting the ultrasonic transducer 5 to the case body 61, HF active electrode terminal 521 is electrically connected to the HF active electrode terminal 162 provided in the terminal unit 14. Because the HF active electrode terminal 521 is provided over the entire circumference in the circumferential direction around the central axis Ax as described above, even when the HF active electrode terminal 162 is rotated around the center axis Ax in response to the rotation control to the rotary knob 9 by an operator, the HF active electrode terminal 162 is always electrically connected to the HF active electrode terminal 521.

The outer peripheral surface of the terminal holding portion 512 also includes US return electrode terminal 522, which is disposed on the proximal end side Ar2 of and separated from the HF active electrode terminal 521. U.S. return electrode terminal 522 is provided over the entire circumference of the terminal holding portion 512 in the circumferential direction around the central axis Ax. Then, by connecting the ultrasonic transducer 5 to the case body 61, U.S. return electrode terminal 522 is electrically connected to the U.S. return electrode terminal 163 provided in the terminal unit 14. Because the U.S. return electrode terminal 522 is provided over the entire circumference in the circumferential direction around the central axis Ax as described above, even when the U.S. return electrode terminal 163 is rotated around the center axis Ax in response to the rotation control to the rotary knob 9 by an operator, the U.S. return electrode terminal 163 is always electrically connected to the U.S. return electrode terminal 522.

The outer peripheral surface of the terminal holding portion 512 also includes US active electrode terminal 523, which is disposed on the most proximal end side Ar2 of the terminals 521 to 523. U.S. active electrode terminal 523 is provided over the entire circumference of the terminal holding portion 512 in the circumferential direction around the central axis Ax. Then, by connecting the ultrasonic transducer 5 with respect to the case body 61, U.S. active electrode terminal 523 is electrically connected to the US active electrode terminal 164 provided in the terminal unit 14. Because U.S. active electrode terminal 523 is provided over the entire circumference of the terminal holding portion 512 in the circumferential direction around the central axis Ax as described above, even when U.S. active electrode terminal 164 is rotated around the central axis Ax in response to the rotation control to the rotary knob 9 by an operator, the U.S. active electrode terminal 164 is always electrically connected to the U.S. active electrode terminal 523.

The ultrasonic vibrator 53 generates ultrasonic vibration under control by the control system 3. In the first embodiment, the ultrasonic vibrator 53 is constituted by a bolt-clamped Langevin transducer (BLT). As shown in FIG. 4, the ultrasonic vibrator 53 comprises a vibrator body 54, a front mass 55, and a back mass 56.

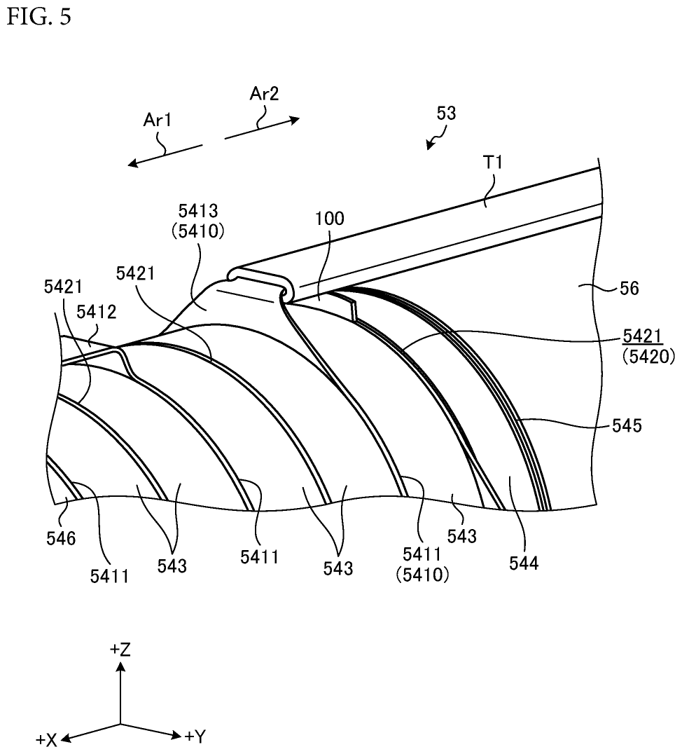

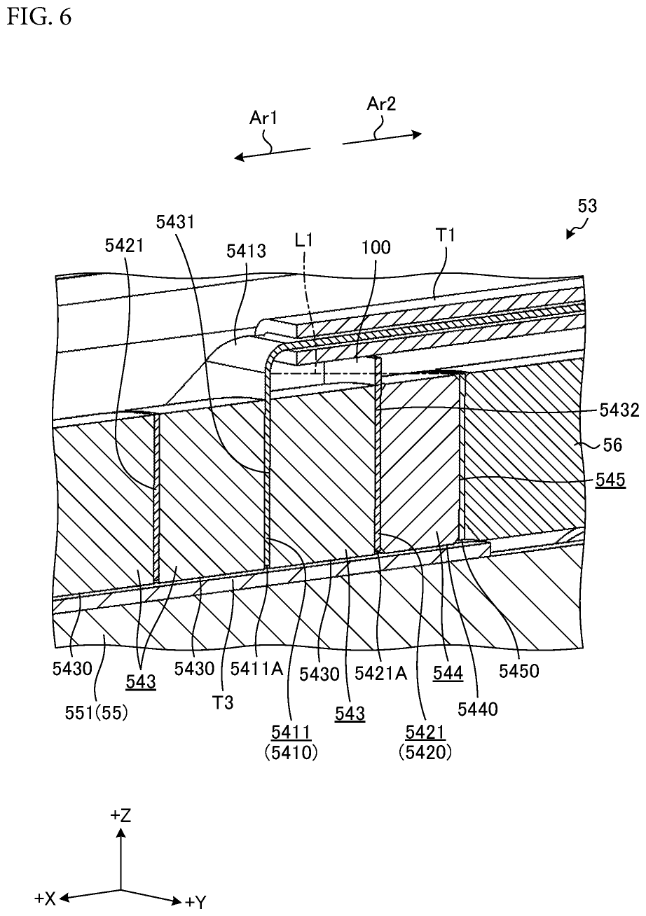

As shown in FIG. 4, the transducer body 54 includes a first electrode unit 541 and a second electrode unit 542, a plurality of piezoelectric elements 543 (four in the present first embodiment), an insulating plate 544, and a third electrode 545. The first electrode unit 541 has a portion where an ultrasonic drive voltage for generating ultrasonic vibration is applied from the control system 3 and that portion a is composed of a conductive material. Also as shown in FIG. 4, the first electrode unit 541 includes a plurality of positive electrode plates 5411 (two in this first embodiment), a positive electrode wiring part 5412 (see FIG. 5), and a positive electrode terminal 5413 (see FIG. 5). Each of the plurality of positive electrode plates 5411 has a disc shape having an opening 5411A in the center (see FIG. 6) and are, respectively, arranged along the central axis Ax. Positive electrode wiring part 5412 is a portion for electrically connecting the outer edge portions of the positive electrode plate 5411 to each other. Positive electrode terminal 5413 extends outward along the radial direction of the positive electrode plate 5411 from the outer edge of the positive electrode plate 5411 located on the most proximal end side Ar2 of the plurality of positive electrode plates 5411. Positive electrode terminal 5413 is bent substantially 90° toward the proximal end side Ar2 and the bent portion extends linearly along the central axis Ax toward the proximal end side Ar2. More specifically, the positive electrode terminal 5413 extends outward along the radial direction of the positive electrode plate 5411 located on the most proximal end side Ar2 of the plurality of positive electrode plates 5411 and the positive electrode terminal 5413 extends radially outward a distance that is further than the outer edge of other of the plurality of positive electrode plates 5411 and also extends radially outward a distance that is greater than the outer circumference side of the piezoelectric elements 543.

Further, the positive electrode plate 5411 and the positive electrode terminal 5413 located on the most proximal end side Ar2 of the plurality of positive electrode plates 5411 correspond to the first electrode 5410 (see FIG. 6) according to the present invention. The positive electrode terminal 5413, by passing through the electrical path provided inside the TD case 51 (not shown), is electrically connected to the U.S. active electrode terminal 523. That is, the first electrode unit 541 is electrically connected to the U.S. active electrode terminal 523.

In the positive electrode terminal 5413, the outer peripheral surface of the portion extending linearly along the central axis Ax toward the proximal end side Ar2 is covered by an insulating tube T1 having an electrically insulating property (see FIG. 5).

The second electrode unit 542 is made of a conductive material and is a portion to which a reference voltage for generating ultrasonic vibration is applied from the control device 3. As shown in FIG. 4, the second electrode unit 542 includes a plurality of negative electrode plates 5421 (three in this first embodiment), a plurality of negative electrode wiring parts 5422 (two in this first embodiment), and a negative electrode terminal 5423.

Each of the plurality of negative electrode plates 5421 has a disc shape having an opening 5421A in the center (see FIG. 6) and are arranged, respectively, along the central axis Ax. The negative electrode plate 5421 located on the most proximal end side Ar2 of the plurality of negative electrode plates 5421 corresponds to the second electrode 5420 (see FIG. 6) according to the present invention.

As shown in FIG. 4, the negative electrode plates 5421 and the positive electrode plates 5411 are arranged alternately along the central axis Ax. In this embodiment, the negative electrode plate 5421 positioned on the proximal end side Ar2 of the plurality of negative electrode plates 5421 is disposed at a position closer to the back mass 56 than the positive electrode plate 5411 positioned on the proximal end side Ar2 of the plurality of positive electrode plates 5411. The outer edge portions of the negative electrode plates 5421 adjacent to each other are electrically connected by one of the plurality of negative electrode wiring parts 5422. Additionally, the negative electrode wiring parts 5422 and the positive electrode wiring parts 5412 are respectively provided at different positions in the circumferential direction around the central axis Ax.

Negative electrode terminal 5423 extends outward along the radial direction of the negative electrode plate 5421 from the outer edge of the negative electrode plate 5421 located on the most proximal end side Ar2 of the plurality of negative electrode plate 5421. Negative electrode terminal 5423 is bent substantially 90° toward the proximal end side Ar2 and the bent portion extends linearly along the central axis Ax toward the proximal end side Ar2. The negative electrode terminal 5423, by passing through the electrical path provided inside the TD case 51 (not shown), is electrically connected to the U.S. return electrode terminal 522. In other words, the second electrode unit 542 is electrically connected to the U.S. return electrode terminal 522.

In the negative electrode terminal 5423, the outer peripheral surface of the portion extending linearly along the central axis Ax toward the proximal end side Ar2 is covered by an insulating tube T2 having an electrically insulating property (FIG. 4).

Additionally, the negative electrode terminal 5423 and the positive electrode terminal 5413 are respectively provided at different positions in the circumferential direction around the central axis Ax.

A plurality of piezoelectric elements 543, each having a cylindrical shape with an opening 5430 in the center (see FIG. 6), are disposed between the negative electrode plate 5421 and the positive electrode plate 5411. That is, a plurality of piezoelectric elements 543 are laminated along the central axis Ax. Then, in response to the ultrasonic drive voltage applied to the first electrode unit 541 and the reference voltage applied to the second electrode unit 542, a plurality of piezoelectric elements 543 alternately and repeatedly expand and shrink along the stacking direction as a result of a potential difference occurring in the stacking direction along the central axis Ax. Thus, the ultrasonic vibrator 53 generates an ultrasonic vibration of the longitudinal vibration in which the stacking direction is the vibration direction.

In the piezoelectric element 543, the end surface in contact with the first electrode 5410 corresponds to the first end surface 5431 (see FIG. 6) according to the present invention. Further, in the piezoelectric element 543, the end surface in contact with the second electrode 5420 corresponds to a second end surface 5432 (see FIG. 6) according to the present invention. The first end surface 5431 and the second end surface 5432 may be constituted by a flat surface perpendicular to the central axis Ax or may be constituted by a curved surface.

Insulating plate 544 has a cylindrical shape with an opening 5440 (see FIG. 6) in the center and is composed of a material having an electrically insulating property. The insulating plate 544 faces the second end surface 5432 across the second electrode 5420 and is disposed in contact with the second electrode 5420.

The third electrode 545 has a disc shape with an opening 5450 (see FIG. 6) in the center and is constructed of a conductive material. The third electrode 545 is positioned facing the second electrode 5420 across the insulating plate 544. The third electrode 545 is electrically connected to the HF active electrode terminal 521 via a electrical path provided inside the TD case 51 (not shown).

The front mass 55 is constructed of an electrically conductive material and has an elongated shape that extends linearly along the central axis Ax. As shown in FIG. 4, the front mass 55 includes a first mounting portion 551, a cross-sectional area changing portion 552, and a second mounting portion 553.

The first mounting portion 551 is in the form of a bolt extending linearly along the central axis Ax. The first mounting portion 551 extends through the opening 5421A of each of the plurality of negative electrode plates 5421, the opening 5411A of each of the plurality of positive electrode plates 5411, and the opening 5430 of each of the plurality of piezoelectric elements 543, respectively. As shown in FIG. 4, the back mass 56 is attached at the end portion of the proximal end side Ar2 of the first mounting portion 551. In some embodiments, the back mass 56 is a nut made of a conductive material. The back mass 56 is electrically connected to the front mass 55. In the first embodiment, in each of the positive electrode plate 5411, the negative electrode plate 5421, the piezoelectric element 543, the insulating plate 544, and the third electrode 545, the outer diameter and inner diameter are set to be substantially the same to each other.

Here, in the outer peripheral surface of the first mounting portion 551, each of the positive electrode plate 5411, the negative electrode plate 5421, the piezoelectric element 543, and the portion facing the insulating plate 544 is covered by an insulating tube T3 having an electrically insulating property (see FIG. 6). The insulating tube T3 prevents a short circuit between the positive electrode plate 5411 and the negative electrode plate 5421 and the first mounting portion 551.

Cross-sectional area changing portion 552 is provided at the end of the distal end side Ar1 of the first mounting portion 551 and is a portion for amplifying the amplitude of the ultrasonic vibration. Further and as shown in FIG. 4, in the cross-sectional area changing portion 552, the end portion of the proximal end side Ar2 has a larger diameter than the first mounting portion 551. Also, the end portion of the distal end side Ar1 of the cross-sectional area changing portion 552 has a truncated cone shape cross-sectional area that is reduced toward the distal end side Ar1. When assembled with the first mounting portion 551 extending along the central axis Ax and through the plurality of positive electrode plates 5411, the plurality of negative electrode plates 5421, the plurality of piezoelectric elements 543, the insulating plate 544, and the third electrode 545, these elements are sandwiched between the cross-sectional area changing portion 552 and the back mass 56 and are integrally fastened in a state having a substantially cylindrical shape. Thus, the third electrode 545 electrically connects with the back mass 56.

In the first embodiment, the insulating plate 546 (FIG. 4) is interposed between the cross-sectional area changing portion 552 and the negative electrode plate 5421 and is located on the most distal end side Ar1 of the plurality of negative electrode plates 5421. The insulating plate 546 is formed of the same insulator material as the insulating plate 544.

As shown in FIG. 4, the second mounting portion 553 is provided at the end of the distal end side Ar1 of the cross-sectional area changing portion 552 and extends linearly along the central axis Ax. Then, the end of the distal end side Ar1 of the second mounting portion 553, by connecting the ultrasonic transducer 5 to the case body 61, is mechanically and electrically connected to the end of the proximal end side Ar2 in the vibration transmission member 13.

The ultrasonic transducer 5 described above includes a short-circuit preventing portion 100 for preventing a short circuit between the first electrode 5410 and the third electrode 545. The detailed configuration of the short-circuit preventing portion 100 will be described in “Configuration of the short-circuit preventing portion” to be described later.

Composition of the Control Device

Control system 3 controls the operation of the ultrasonic treatment instrument 2. Specifically, the control system 3, by passing operating signals through the electrical cable C (FIG. 1), detects the operation of the switch 8 by an operator. Then, when the control system 3 detects the switch operation, operating signals and power are passed through the electric cable C to impart treatment energy to the target site grasped between the jaw 12 and the treatment portion 131. Specifically, the control system 3 imparts procedure energy to the target site grasped between the jaw 12 and the treatment portion 131 by passing through the electric cable C. In other words, the jaws 12 and the treatment portion 131 correspond to the end effectors 17 (FIGS. 1 and 2) according to the present invention.

For example, when applying ultrasonic energy to the target site, the control system 3 supplies drive power to the U.S. active electrode terminal 164,523 or the like, to apply an ultrasonic drive voltage to the first electrode unit 541 by passing it through the electrical cable C. Further, the control system 3 applies a reference voltage to the second electrode unit 542 by passing through the electrical cable C and U.S. return electrode terminal 163,522 or the like. Thus, the ultrasonic vibrator 53 generates a longitudinal vibration (ultrasonic vibration) which vibrates in a direction along the central axis Ax. The treatment portion 131 also vibrates at a desired amplitude by the longitudinal vibration. Then, an ultrasonic vibration is applied from the treatment portion 131 to the target site grasped between the jaw 12 and the treatment portion 131. In other words, ultrasonic energy is applied from the treatment portion 131 to the target site.

Further, for example, when high-frequency energy is applied to the target site, the control system 3 supplies high-frequency power to the jaw 12 by following the conductive path of the electrical cables C to HF return electrode terminals 161, 1121 to the electrical path 1122 to the shaft 10 and supplies high-frequency power to the vibration transmission member 13 by following the conductive path of the electrical cables C to HF active electrode terminals 162,521 to the third electrodes 545 to the back mass 56 to the front mass 55. Thus, a high frequency current flows through the target site grasped between the jaw 12 and the treatment portion 131. In other words, the subject site is imparted with high frequency energy.

Configuration of the Short-Circuit Prevention Portion

Next, the configuration of the short-circuit preventing portion 100 is described. FIGS. 5 and 6 are diagrams illustrating the configuration of the short-circuit preventing portion 100. Specifically, FIG. 5 is a perspective view showing an end portion of the proximal end side Ar2 of the ultrasonic vibrator 53. FIG. 6 is a cross-sectional view formed by cutting the ultrasonic vibrator 53 through the positive electrode terminal 5413 by a plane including the central axis Ax.

In the first embodiment, the short-circuit preventing portion 100 prevents a short circuit between the third electrode 545 and the positive electrode plate 5411 on the outer peripheral surface side of the third electrode 545. Also, the short-circuit prevention portion 100 is a portion of the second electrode 5420 as shown in FIG. 5 or FIG. 6.

Specifically, the short-circuit prevention portion 100 is a portion protruding outwardly along the radial direction of the second electrode 5420 from a portion of an outer edge of the second electrode 5420 as shown in FIG. 5 and FIG. 6. The short-circuit preventing portion 100, with respect to the positive electrode terminal 5413, is provided at the same position in the circumferential direction around the central axis Ax.

The short-circuit preventing portion 100 is located between the positive electrode terminal 5413 and the third electrode 545 and projects past an outermost surface of the electrode toward the positive electrode terminal 5413. For example, the short-circuit preventing portion 100 is located between piezoelectric element 543 and insulating plate 544 and the short-circuit preventing portion 100 projects past the surfaces of the piezoelectric element 543 and insulating plate 544. Because of this projecting, the short-circuit preventing portion 100 intersects an imaginary straight line L1 (FIG. 6), where the imaginary straight line L1 is an imaginary straight line between the outermost side of the third electrode 545 and the first electrode 5410 (where the imaginary straight line L1 does not have a circumferential component, i.e., in a top view, the imaginary straight line L1 without circumferential component is parallel to the central axis Ax). In various embodiments, the imaginary straight line L1 is any one of the various straight lines without circumferential component between the outermost side of the third electrode 545 and a portion of the first electrode 5410 which protrudes further than the outermost side of the third electrode 545 (such as a portion of the positive electrode terminal 5413). In some embodiments, the imaginary straight line L1 without circumferential component is the shortest imaginary straight line having a first end at an apex of the bend in the first electrode 5410 where the positive electrode plate 5411 transitions to become the positive electrode terminal 5413 and a second end at the outermost side of the third electrode 545. In other embodiments, the imaginary straight line L1 without circumferential component is the shortest imaginary straight line having a first end at any location within the bend in the first electrode 5410 where the positive electrode plate 5411 transitions to become the positive electrode terminal 5413 and a second end at the outermost side of the third electrode 545. In further embodiments, the imaginary straight line L1 is the shortest imaginary straight line without circumferential component between the outermost side of the third electrode 545 and any uncoated location on the first electrode 5410, e.g., a location on the first electrode 5410 that is not covered by insulating tube T1, such as a portion of positive electrode plate 5411 or a portion of the positive electrode terminal 5413. The short-circuit preventing portion 100 can be a portion of or can be associated with second electrode 5420 located between piezoelectric element 543 and insulating plate 544 or the short-circuit preventing portion 100 can be a separate element located between piezoelectric element 543 and insulating plate 544.

According to the first embodiment described above, the following effects can be achieved. The ultrasonic transducer 5 according to the first embodiment is a part of the second electrode 5420 and includes a short circuit prevention portion 100 disposed on the straight line L1. That is, a portion of the second electrode 5420 is disposed in a state of blocking between the first electrode 5410 and the third electrode 545. Therefore, according to the ultrasonic transducer 5 of the first embodiment, when a short circuit occurs between the first electrode 5410 and the second electrode 5420, a short circuit between the first electrode 5410 and the third electrode 545 can be prevented more reliably. Specifically, for example, when a short circuit is made between the first electrode 5410 and the third electrode 545, the ultrasonic energy to be applied to the piezoelectric element 543 is transmitted to the patient following a path that imparts high frequency energy including the third electrode 545. This provides the patient with excessive energy. In the configuration according to the first embodiment, a short circuit between the first electrode 5410 and the third electrode 545 can be prevented. Although, a short circuit is caused between the first electrode 5410 and the second electrode 5420, the short circuit between the first electrode 5410 and the second electrode 5420 does not provide excessive energy to the patient, and the short circuit detection is activated in the control system 3. In this case, the operator or the like can safely continue the operation by performing an appropriate response, such as replacing the ultrasonic transducer 5. Incidentally, in the case of a short-circuit between the second electrode 5420 and third electrode 545, since the second electrode 5420 is an electrode to which a reference voltage is applied, it is the same as described above that no excessive energy is applied.

### Modified Example 1-1

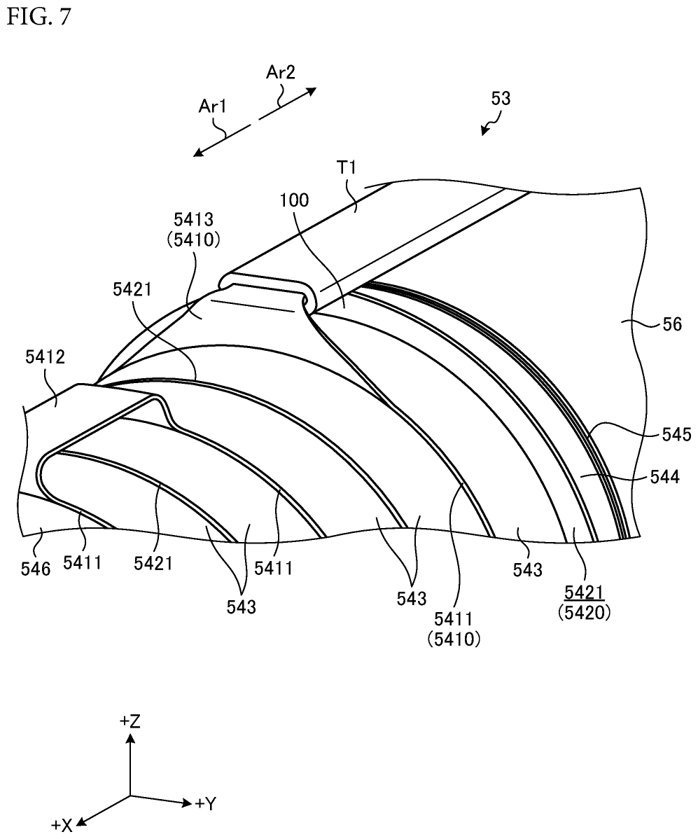

FIG. 7 is a diagram illustrating a modification 1-1 of the first embodiment; Specifically, FIG. 7 is a diagram corresponding to FIG. 5.

According to the present modification 1-1 shown in FIG. 7, instead of the short-circuit preventing portion 100 as described in the first embodiment, a short-circuit preventing portion 100 of modification 1-1 shown in FIG. 7 may be adopted. Specifically, the short circuit preventing portion 100 according to modification 1-1 is a portion protruding outward along the radial direction of the second electrode 5420 from the entire outer edge of the second electrode 5420. That is, the short-circuit preventing portion 100 according to the present modification 1-1 extends over the entire circumference in the circumferential direction around the central axis Ax.

According to modification 1-1 described above, by increasing the area in which the short circuit prevention portion 100 functions, it is possible to further and reliably prevent the short circuit between the first electrode 5410 and the third electrode 545.

### Modified Example 1-2

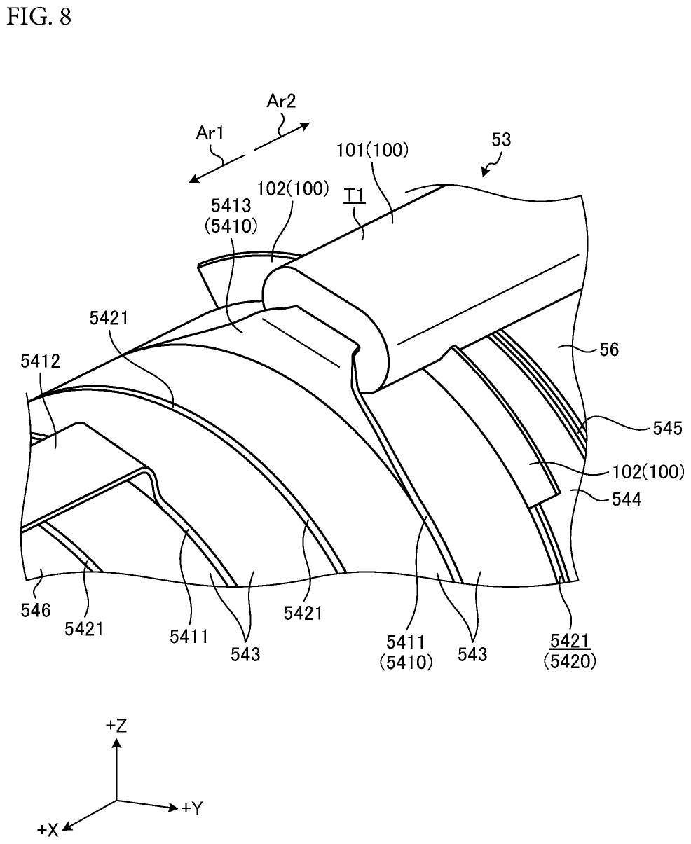

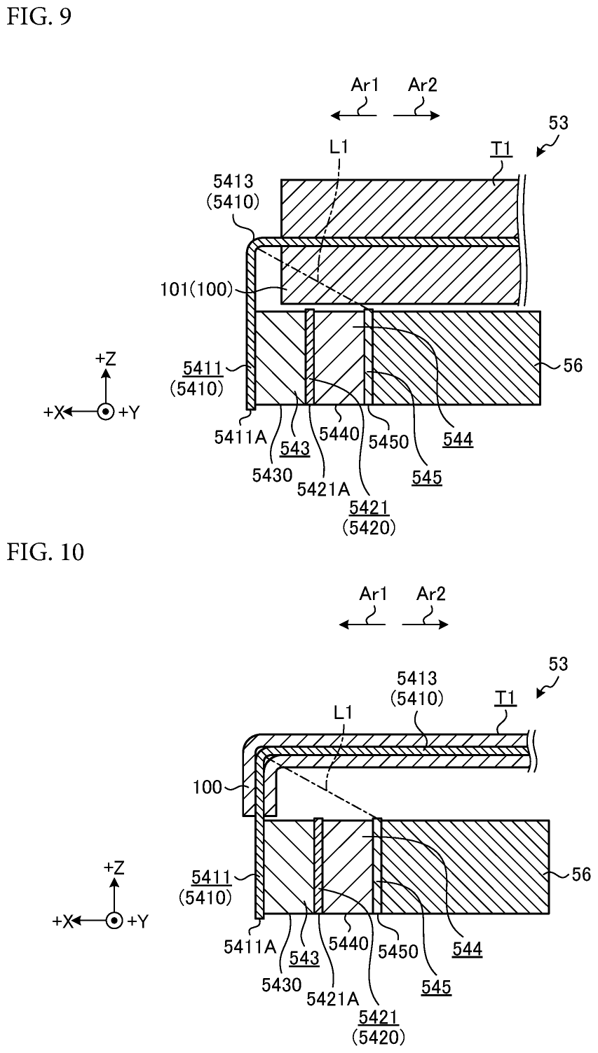

FIGS. 8 and 9 are views illustrating modification 1-2 of the first embodiment. Specifically, FIG. 8 is a view corresponding to FIG. 5 and FIG. 9 is a cross-sectional view corresponding to FIG. 6. According to the present modification 1-2 shown in FIGS. 8 and 9, instead of the short circuit preventing portion 100 as described in the first embodiment, a short circuit preventing portion 100 of modification 1-2 shown in FIGS. 8 and 9 may be adopted. The short-circuit preventing portion 100 according to the present modification 1-2 includes a first short-circuit preventing portion 101 which is a part of the insulating tube T1, and a second short-circuit preventing portion 102 which is a part the second electrode 5420. Specifically and as shown in FIG. 8 or FIG. 9, according to the present modification 1-2, the insulating tube T1 intersects the imaginary straight line L1 (FIG. 9), where the imaginary straight line L1 is the longest imaginary straight line between the outermost side of the third electrode 545 and the first electrode 5410. The portion of the insulating tube T1 that intersects the imaginary straight line L1 has a thickness dimension that is larger than the thickness dimension of the insulating tube T1 described in the first embodiment. As a result of the thicker dimension of the insulating tube T1 at the noted location, the portion of the insulating tube T1 becomes a first short-circuit preventing portion 101.

Further and as shown in FIG. 8, the second short-circuit preventing part 102 includes a portion protruding outward along the radial direction of the second electrode 5420 further than portions of the second electrode 5420. The protruding portions of the second short-circuit preventing part 102 are on either side (in the circumferential direction) of the insulating tube T1. By having protruding portions of the second short-circuit preventing part 102 that extend a longer length in the circumferential direction than in the first embodiment, the second short-circuit preventing portion 102 can be provided at different positions in the circumferential direction around the central axis Ax as compared to the positive electrode terminal 5413.

According to modification 1-2 described above, by increasing the area in which the short circuit prevention portion 100 functions, it is possible to further and reliably prevent the short circuit between the first electrode 5410 and the third electrode 545.

### Modified Example 1-3

FIG. 10 is a diagram illustrating modification 1-3 of the first embodiment. Specifically, FIG. 10 is a cross-sectional view corresponding to FIG. 6. In FIG. 10, the configuration of the +Z-axis side along the central axis Ax of the ultrasonic vibrator 53 from the distal end side Ar1 of the first electrode 5410 is illustrated, and other features of the ultrasonic vibrator 53 are omitted.

According to the present modification 1-3 shown in FIG. 10, instead of the short circuit preventing portion 100 as described in the first embodiment, the short circuit preventing portion 100 of modification 1-3 shown in FIG. 10 may be adopted. Short-circuit preventing portion 100 according to the present modification 1-3 is a part of the insulating tube T1. Specifically, according to modification 1-3 the insulating tube T1 in the positive electrode terminal 5413 is extended and, in addition to a portion extending linearly along the central axis Ax toward the proximal end side Ar2 and covering the bent portion, a portion of the insulating tube T1 also covers the portion of the positive electrode terminal 5413 that extends outward along the radial direction of the positive electrode plate 5411 from the outer edge of the positive electrode plate 5411 (hereinafter, described as a protruding portion). As a result, a portion of the insulating tube T1 covering the protruding portion of the positive electrode terminal 5413 intersects the imaginary straight line L1 (FIG. 10), where the imaginary straight line L1 is the longest imaginary straight line between the outermost side of the third electrode 545 and the first electrode 5410. Then, a part of the insulating tube T1 is a short-circuit preventing portion 100.

Even when a part of the insulating tube T1 is set as the short circuit preventing portion 100 as in modification 1-3 described above, it is possible to more reliably prevent a short circuit between the first electrode 5410 and the third electrode 545 in the same manner as in the first embodiment described above.

### Modified Example 1-4

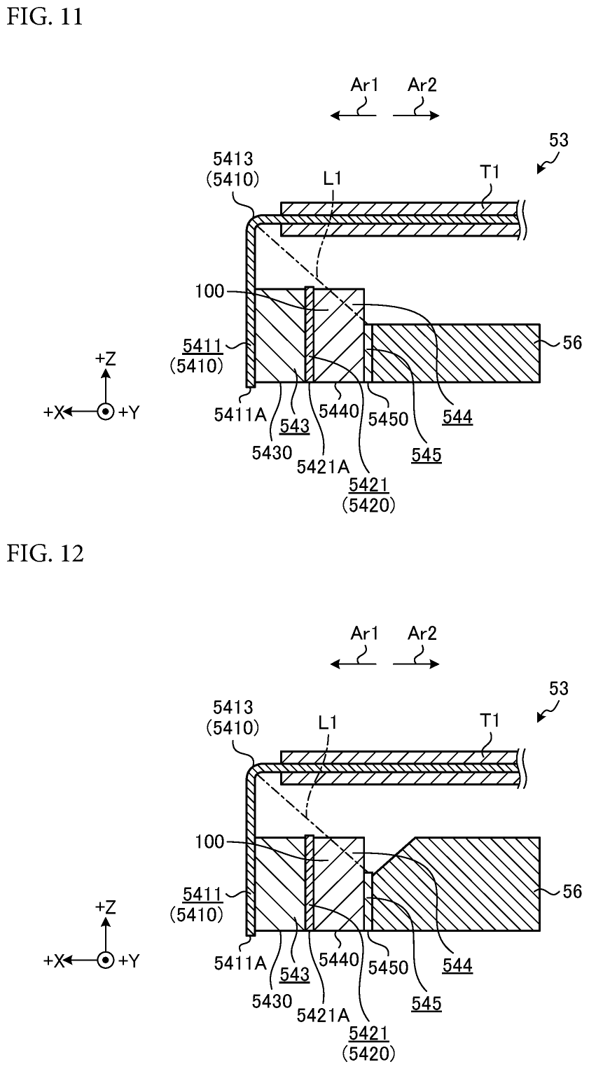

FIG. 11 is a diagram illustrating modification 1-4 of the first embodiment. Specifically, FIG. 11 is a cross-sectional view corresponding to FIG. 6. In FIG. 11, the configuration of the +Z-axis side along the central axis Ax of the ultrasonic vibrator 53 from the distal end side Ar1 of the first electrode 5410 is illustrated, and other features of the ultrasonic vibrator 53 are omitted.

According to the present modification 1-4 shown in FIG. 11, instead of the short-circuit preventing portion 100 as described in the first embodiment, the short-circuit preventing portion 100 of modification 1-4 may be adopted. Short-circuit preventing portion 100 according to the present modification 1-4 is a part of the insulating plate 544. Specifically, the outer diameter of the third electrode 545 and at least a portion of the back mass 56 are smaller than the outer diameters of the positive electrode plate 5411, the piezoelectric element 543, the second electrode 5420, and the insulating plate 544. As a result, a portion of the insulating plate 544 intersects the imaginary straight line L1 (FIG. 11), where the imaginary straight line L1 is the longest imaginary straight line between the outermost side of the third electrode 545 and the first electrode 5410. Then, a part of the insulating plate 544 is a short-circuit preventing portion 100.

Even in the case where a part of the insulating plate 544 is the short-circuit preventing portion 100 as in modification 1-4, the short-circuit between the first electrode 5410 and the third electrode 545 can be more reliably prevented similarly to the first embodiment described above.

### Modified Example 1-5

FIG. 12 is a diagram illustrating modification 1-5 of the first embodiment. Specifically, FIG. 12 is a cross-sectional view corresponding to FIG. 6. In FIG. 12, the configuration of the +Z-axis side along the central axis Ax of the ultrasonic vibrator 53 from the distal end side Ar1 of the first electrode 5410 is illustrated, and other features of the ultrasonic vibrator 53 are omitted.

Although according to modification 1-4, the outer diameter dimension of the back mass 56 is the same as the outer diameter dimension of the third electrode 545, the outer diameter dimension of the back mass 56 is not limited thereto. Thus, according to the present modification 1-5, the outer diameter of the back mass 56 may be larger than the outer diameter dimension of the third electrode 545. In a specific embodiments according to the modification 1-5 and as shown in FIG. 12, the outer diameter of the back mass 56 can be the same as the outer diameter of the positive electrode plate 5411, the piezoelectric element 543, the second electrode 5420, and the insulating plate 544 and the back mass 56 may further have a chamfered corner portion at the tip of the outer peripheral edge on the distal end side Ar1. As in modification 1-4, in modification 1-5 a portion of the insulating plate 544 intersects the imaginary straight line L1 (FIG. 12), where the imaginary straight line L1 is the longest imaginary straight line between the outermost side of the third electrode 545 and the first electrode 5410. Then, a part of the insulating plate 544 is a short-circuit preventing portion 100.

### Modified Example 1-6

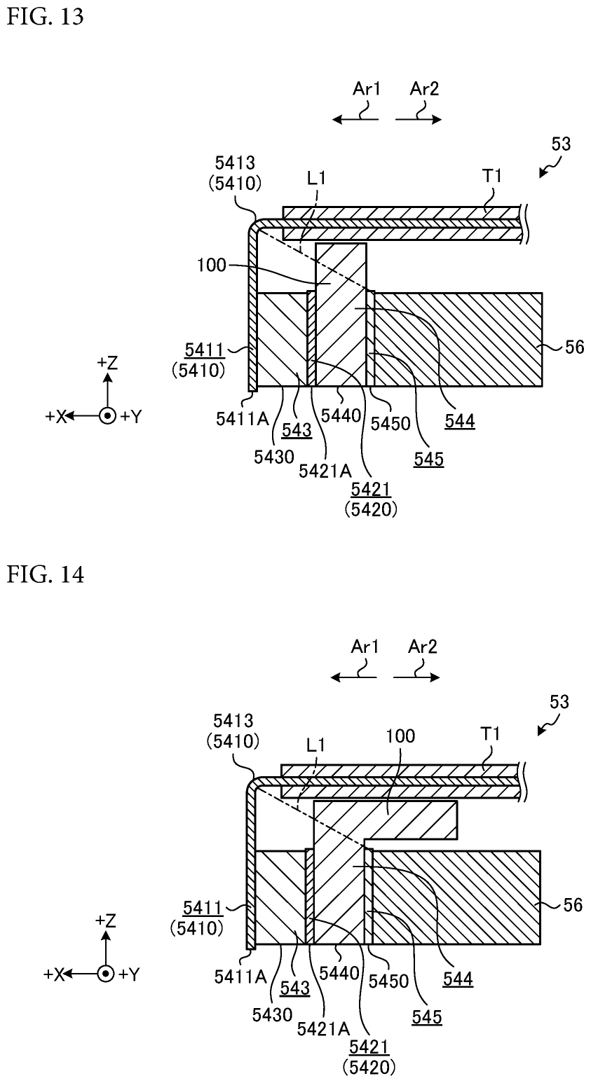

FIG. 13 is a diagram illustrating modification 1-6 of the first embodiment. Specifically, FIG. 13 is a cross-sectional view corresponding to FIG. 6. In FIG. 13, the configuration of the +Z-axis side along the central axis Ax of the ultrasonic vibrator 53 from the distal end side Ar1 of the first electrode 5410 is illustrated, and other features of the ultrasonic vibrator 53 are omitted.

According to the present modification 1-6 shown in FIG. 13, instead of the short-circuit preventing portion 100 described in the first embodiment, the short circuit preventing portion 100 of modification 1-6 shown in FIG. 13 may be adopted. Short-circuit preventing portion 100 according to the present modification 1-6 is a part of the insulating plate 544. Specifically, according to modification 1-6 the outer diameter of the insulating plate 544 is larger than the outer diameter of the positive electrode plate 5411, the piezoelectric element 543, the second electrode 5420, and the third electrode 545. As a result, a portion of the insulating plate 544 intersects the imaginary straight line L1 (FIG. 13), where the imaginary straight line L1 is the longest imaginary straight line between the outermost side of the third electrode 545 and the first electrode 5410. Then, a part of the insulating plate 544 is a short-circuit preventing portion 100. As a further modification, the short-circuit preventing portion 100 according to the modification 1-6 may be configured to extend the entire circumference in the circumferential direction around the central axis Ax, or may be configured to be provided only in a portion of the position in the circumferential direction.

Even when a part of the insulating plate 544 is the short-circuit preventing portion 100 as in modification 1-6 described above, the short-circuit between the first electrode 5410 and the third electrode can be more reliably prevented similarly to the first embodiment described above.

### Modified Example 1-7

FIG. 14 is a diagram illustrating modification 1-7 of the first embodiment. Specifically, FIG. 14 is a cross-sectional view corresponding to FIG. 6. In FIG. 14, the configuration of the +Z-axis side along the central axis Ax of the ultrasonic vibrator 53 from the distal end side Ar1 of the first electrode 5410 is illustrated, and other features of the ultrasonic vibrator 53 are omitted.

Modification 1-7 is similar to modification 1-6, but as shown in FIG. 14, the structure of the short-circuit preventing portion 100 in modification 1-7 includes a structure on the insulating plate 544 where an outer edge portion of the insulating plate 544 is bent substantially 90° and extends along the central axis Ax direction toward the proximal end side Ar2, which creates an L-shaped cross section for the insulating plate 544. As a result, a portion of the insulating plate 544 intersects the imaginary straight line L1 (FIG. 14), where the imaginary straight line L1 is the longest imaginary straight line between the outermost side of the third electrode 545 and the first electrode 5410. Then, a part of the insulating plate 544 is a short-circuit preventing portion 100.

As a further modification, the short-circuit preventing portion 100 according to the modification 1-7 may be configured to extend the entire circumference in the circumferential direction around the central axis Ax, or may be configured to be provided only in a portion of the position in the circumferential direction. Further, as the short-circuit preventing portion 100, only a portion extending in a direction orthogonal to the central axis Ax extends in the entire circumferential direction centered on the central axis Ax and the portion extending in a bent state substantially 90° toward the proximal end side Ar2 may be provided only at a portion of the circumferential direction.

According to modification 1-7 described above, since the minimum distance between conductive parts along the surface of the insulator can be sufficiently secured with respect to the 1-6 modification described above, it is possible to further reliably prevent the short circuit between the first electrode 5410 and the third electrode 545.

### Modified Example 1-8

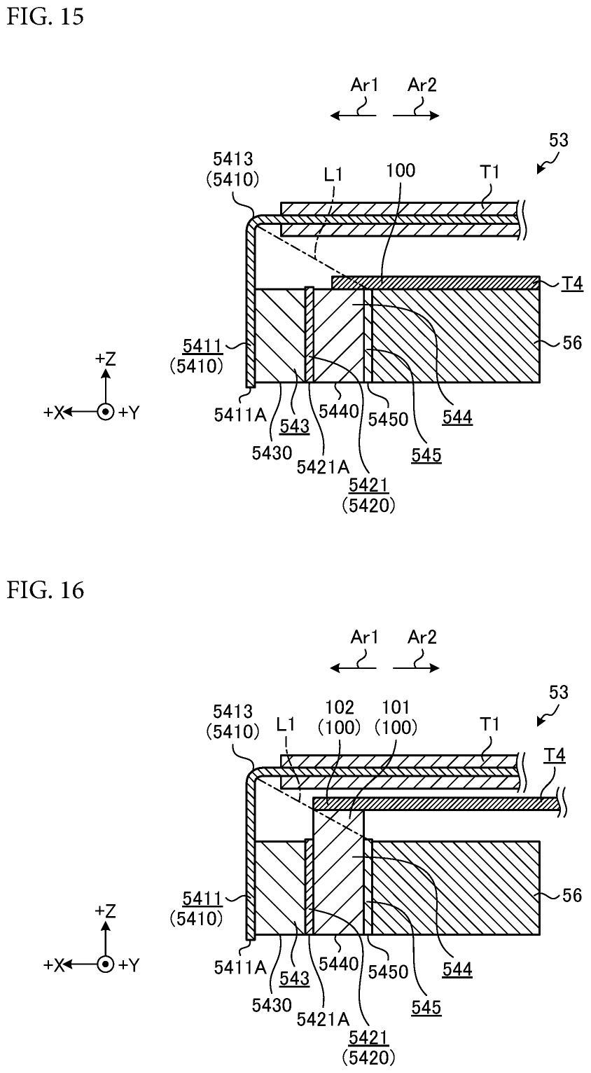

FIG. 15 is a diagram illustrating modification 1-8 of the first embodiment. Specifically, FIG. 15 is a cross-sectional view corresponding to FIG. 6. In FIG. 15, the configuration of the +Z-axis side along the central axis Ax of the ultrasonic vibrator 53 from the distal end side Ar1 of the first electrode 5410 is illustrated, and other features of the ultrasonic vibrator 53 are omitted.

According to the present modification 1-8 shown in FIG. 15, instead of the short circuit preventing portion 100 as described in the first embodiment, the short circuit preventing portion 100 of modification 1-8 shown in FIG. 15 may be adopted. Short-circuit preventing portion 100 according to the present modification 1-8 is a part of the insulating tube T4. Specifically, the insulating tube T4 is composed of a material having an electrical insulating property and, as shown in FIG. 15, the insulating tube T4 extends to cover the outer peripheral surface of at least a portion of the insulating plate 544, the third electrode 545, and the back mass 56. As a result, a portion of the insulating tube T4 intersects the imaginary straight line L1 (FIG. 15), where the imaginary straight line L1 is the longest imaginary straight line between the outermost side of the third electrode 545 and the first electrode 5410. Then, a part of the insulating tube T4 becomes a short-circuit preventing portion 100.

Even when a part of the insulating tube T4 is set as the short circuit preventing portion 100 as in modification 1-8 described above, it is possible to more reliably prevent a short circuit between the first electrode 5410 and the third electrode 545 in the same manner as in the first embodiment described above.

### Modified Example 1-9

FIG. 16 is a diagram illustrating modification 1-9 of the first embodiment. Specifically, FIG. 16 is a cross-sectional view corresponding to FIG. 6. In FIG. 16, the configuration of the +Z-axis side along the central axis Ax of the ultrasonic vibrator 53 from the distal end side Ar1 of the first electrode 5410 is illustrated, and other features of the ultrasonic vibrator 53 are omitted.

Modification 1-9 is similar to modification 1-8, but as shown in FIG. 16, the outer diameter dimension of the insulating plate 544 is larger than the outer diameter dimension of the positive electrode plate 5411, the piezoelectric element 543, the second electrode 5420, and the third electrode 545 and also the insulating tube T4 is in contact with the outer peripheral surface of the insulating plate 544 and there is a gap between the inner peripheral surface of the insulating tube T4 and the outer peripheral surface of the third electrode 545 and the back mass 56. As a result, a portion of the insulating plate 544 and, optionally, a portion of the insulating tube T4, intersects the imaginary straight line L1 (FIG. 16), where the imaginary straight line L1 is the longest imaginary straight line between the outermost side of the third electrode 545 and the first electrode 5410. Then, the short-circuit preventing portion 100 includes a first short-circuit preventing portion 101 that is an outer edge portion of the insulating plate 544 and (optionally) a second short-circuit preventing portion 102 that is a part of the insulating tube T4.

According to modification 1-9 and as compared to modification 1-8, with modification 1-9 it is possible to avoid friction between the insulating tube T4 and the outer peripheral surface of the third electrode 545 and the back mass 56 during the driving of the ultrasonic vibrator 53. Therefore, it is possible to avoid unnecessarily generating heat.

### Modified Example 1-10

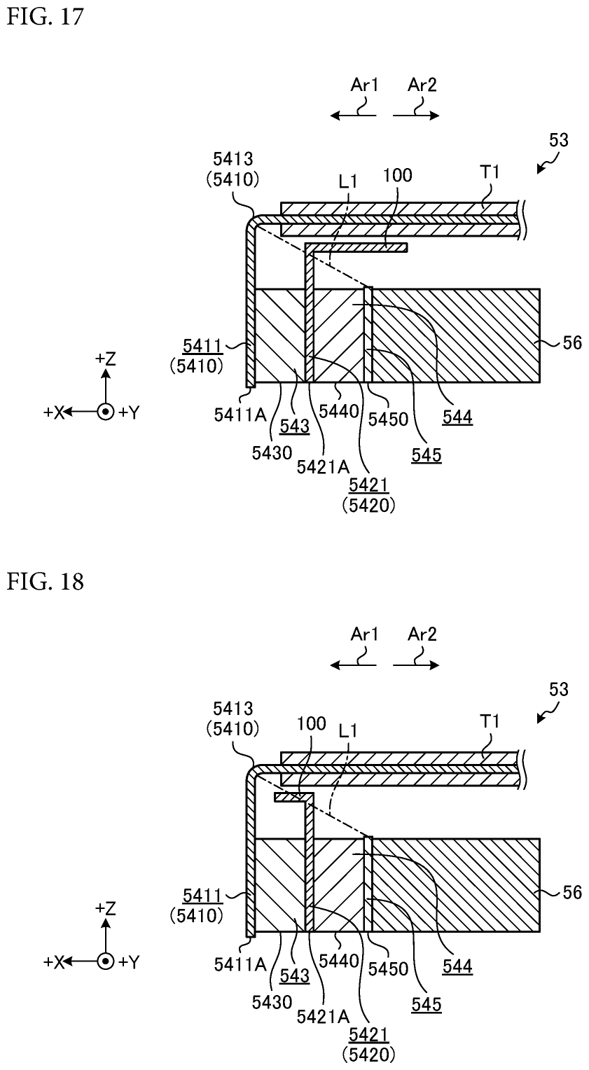

FIG. 17 is a diagram illustrating modification 1-10 of the first embodiment. Specifically, FIG. 17 is a cross-sectional view corresponding to FIG. 6. In FIG. 17, the configuration of the +Z-axis side along the central axis Ax of the ultrasonic vibrator 53 from the distal end side Ar1 of the first electrode 5410 is illustrated, and other features of the ultrasonic vibrator 53 are omitted.

According to the present modification 1-10 shown in FIG. 17, instead of the short circuit preventing portion 100 as described in the first embodiment, the short circuit preventing portion 100 of modification 1-10 shown in FIG. 17 may be adopted. Modification 1-10 is similar to the first embodiment, but as shown in FIG. 17, in addition to the portion protruding outwardly along the radial direction of the second electrode 5420 from a portion of an outer edge of the second electrode 542, the short-circuit prevention portion 100 also includes a portion of the second electrode 5420 extended in a bent state substantially 90° that extends toward the proximal end side Ar2 along the outer edge portion of the second electrode 5420 and forming a structure having an L-shaped cross section. The portion in a bent state extends toward the proximal end side Ar2 to cover at least a portion of the outer peripheral edge of the third electrode 545. As a result, a portion of the second electrode 5420 intersects the imaginary straight line L1 (FIG. 17), where the imaginary straight line L1 is the longest imaginary straight line between the outermost side of the third electrode 545 and the first electrode 5410. Then, the second electrode 5420 is the short-circuit preventing portion 100.

As a further modification, the short-circuit preventing portion 100 according to the modification 1-10 may be configured to extend the entire circumference in the circumferential direction around the central axis Ax, or may be configured to be provided only in a portion of the position in the circumferential direction. Further, as the short-circuit preventing portion 100, only a portion extending in a direction orthogonal to the central axis Ax extends in the entire circumferential direction centered on the central axis Ax and the portion extending in a bent state substantially 90° toward the proximal end side Ar2 may be provided only at a portion of the circumferential direction.

According to modification 1-10, since the structure is provided in which the short circuit between the first electrode 5410 and the second electrode 5420 is positively performed, it is possible to further reliably prevent the short circuit between the first electrode 5410 and the third electrode 545.

### Modified Example 1-11

FIG. 18 is a diagram illustrating modification 1-11 of the first embodiment. Specifically, FIG. 18 is a cross-sectional view corresponding to FIG. 6. In FIG. 18, the configuration of the +Z-axis side along the central axis Ax of the ultrasonic vibrator 53 from the distal end side Ar1 of the first electrode 5410 is illustrated, and other features of the ultrasonic vibrator 53 are omitted.

Modification 1-11 is similar to modification 1-10, but as shown in FIG. 18, the portion of the second electrode 5420 extended in a bent state extends toward the distal end side Ar1 along the outer edge portion of piezoelectric element 543 and forming a structure having an L-shaped cross section. The portion in a bent state extends toward the distal end side Ar1 to cover at least a portion of the outer peripheral edge of the piezoelectric element 543 and to cover at least a portion of the entire circumference. As a result, a portion of the second electrode 5420 intersects the imaginary straight line L1 (FIG. 18), where the imaginary straight line L1 is the longest imaginary straight line between the outermost side of the third electrode 545 and the first electrode 5410. Then, the second electrode 5420 is the short-circuit preventing portion 100.

As a further modification, the short-circuit preventing portion 100 according to the modification 1-11 may be configured to extend the entire circumference in the circumferential direction around the central axis Ax, or may be configured to be provided only in a portion of the position in the circumferential direction. Further, as the short-circuit preventing portion 100, only a portion extending in a direction orthogonal to the central axis Ax extends in the entire circumferential direction centered on the central axis Ax and the portion extending in a bent state substantially 90° toward the distal end side Ar1 may be provided only at a portion of the circumferential direction.

According to modification 1-11, since the short circuit between the first electrode 5410 and the second electrode 5420 is positively performed, it is possible to further reliably prevent the short circuit between the first electrode 5410 and the third electrode 545.

### Modified Example 1-12

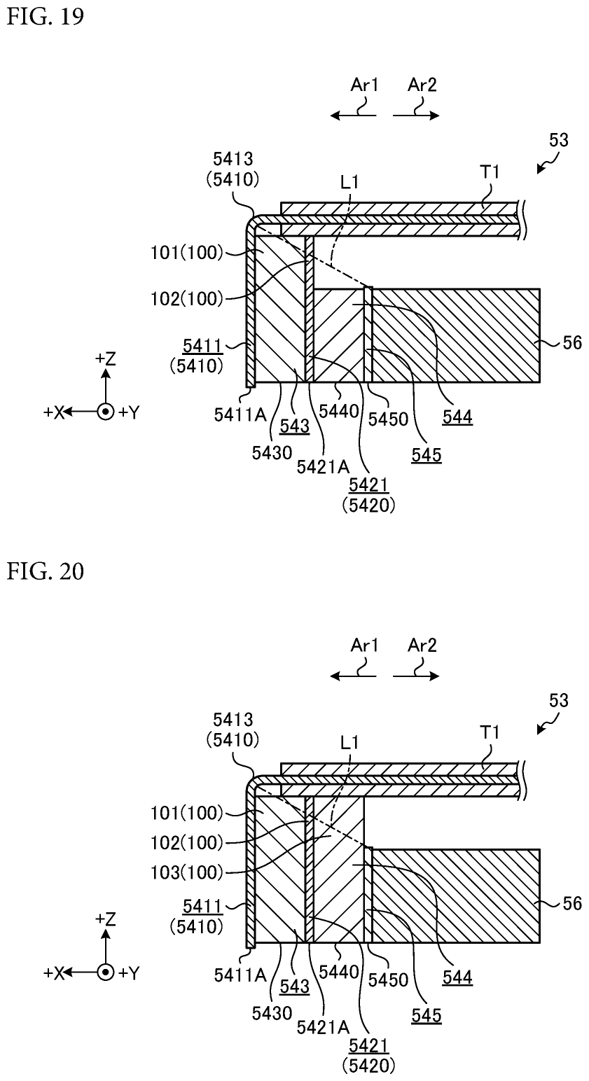

FIG. 19 is a diagram illustrating modification 1-12 of the first embodiment. Specifically, FIG. 19 is a cross-sectional view corresponding to FIG. 6. In FIG. 19, the configuration of the +Z-axis side along the central axis Ax of the ultrasonic vibrator 53 from the distal end side Ar1 of the first electrode 5410 is illustrated, and other features of the ultrasonic vibrator 53 are omitted.

According to the present modification 1-12 shown in FIG. 19, instead of the short circuit preventing portion 100 as described in the first embodiment, the short circuit preventing portion 100 of modification 1-12 shown in FIG. 19 may be adopted. Short-circuit preventing portion 100 according to the present modification 1-12 includes a first short-circuit preventing portion 101 which is a part of the piezoelectric element 543, and a second short-circuit preventing portion 102 which is a part of the second electrode 5420. Specifically and as shown in FIG. 19, in modification 1-12 the outer diameter dimension of the piezoelectric element 543 and the second electrode 5420 are larger than the outer diameter dimension of the positive electrode plate 5411, the insulating plate 544, the third electrode 545, and the back mass 56. As a result, a portion of the piezoelectric element 543 and of the second electrode 5420, respectively, intersect the imaginary straight line L1 (FIG. 19), where the imaginary straight line L1 is the longest imaginary straight line between the outermost side of the third electrode 545 and the first electrode 5410. Then, a part of the piezoelectric element 543 becomes a first short-circuit preventing portion 101 and a portion of the second electrode 5420 becomes a second short-circuit prevention portion 102.

The short-circuit preventing portion 100 according to the modification 1-12 may be configured to extend the entire circumference in the circumferential direction around the central axis Ax, or may be configured to be provided only in a portion of the position in the circumferential direction.

According to the modification 1-12, in addition to a part of the second electrode 5420, since a part of the piezoelectric element 543 is the short-circuit preventing portion 100, a short circuit between the first electrode 5410 and the third electrode 545 can be more reliably prevented.

### Modified Example 1-13

FIG. 20 is a diagram illustrating modification 1-13 of the first embodiment. Specifically, FIG. 20 is a cross-sectional view corresponding to FIG. 6. In FIG. 20, the configuration of the +Z-axis side along the central axis Ax of the ultrasonic vibrator 53 from the distal end side Ar1 of the first electrode 5410 is illustrated, and other features of the ultrasonic vibrator 53 are omitted.

Modification 1-13 is similar to modification 1-12, but as shown in FIG. 20, in addition to the outer diameter dimension of the piezoelectric element 543 and the second electrode 5420 being larger than the outer diameter dimension of the third electrode 545 and the back mass 56, the outer diameter dimension of the insulating plate 544 is also larger than the outer diameter dimension of the third electrode 545 and the back mass 56.

As a result, in addition to a portion of the piezoelectric element 543 and the second electrode 5420, a portion of the insulating plate 544 also intersects the imaginary straight line L1 (FIG. 20), where the imaginary straight line L1 is the longest imaginary straight line between the outermost side of the third electrode 545 and the first electrode 5410. Then, the short-circuit preventing portion 100 according to modification 1-13 includes a first short-circuit preventing portion 101 which is the part in the piezoelectric element 543, a second short-circuit preventing portion 102 which is the part in the second electrode 5420, and a third short-circuit preventing portion 103, which is the part in the insulating plate 544.

The short-circuit preventing portion 100 according to the modification 1-13 may be configured to extend the entire circumference in the circumferential direction around the central axis Ax, or may be configured to be provided only in a portion of the position in the circumferential direction.

According to modification 1-13, in addition to a part of the second electrode 5420 and a part of the piezoelectric element 543, since a part of the insulating plate 544 is the short-circuit preventing portion 100, a short circuit between the first electrode 5410 and the third electrode 545 can be more reliably prevented.

According to the modification 1-13, in addition to a part of the second electrode 5420, since a part of the piezoelectric element 543 is the short-circuit preventing portion 100, a short circuit between the first electrode 5410 and the third electrode 545 can be more reliably prevented.

### Second Embodiment

Next, a second embodiment will be described. In the following description, the same reference numerals will be used for the same configurations as in the first embodiment described above, and detailed description thereof will be omitted or simplified.

In the first embodiment described above, short-circuit preventing portion 100 prevents a short circuit between the first electrode 5410 and the third electrode 545, such as a short circuit between positive electrode plate 5411 and the outer peripheral surface side of the third electrode 545. In contrast, in the short-circuit preventing portion 100 according to the second embodiment, the short-circuit preventing portion 100 prevents a short circuit between the first electrode 5410 and the third electrode 545, such as a short circuit between positive electrode plate 5411 and the inner peripheral surface side of the third electrode 545.

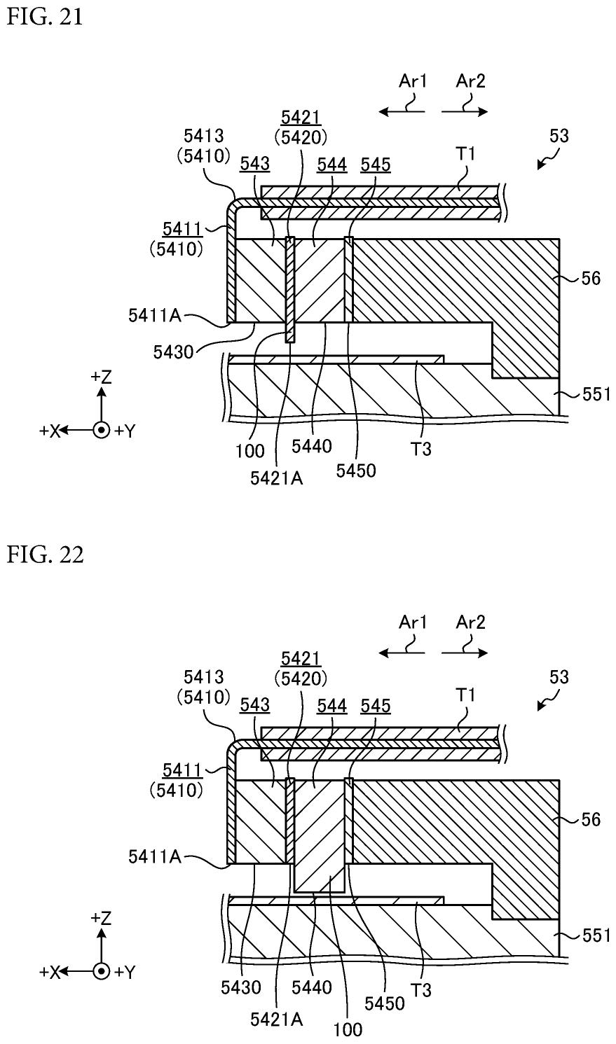

FIG. 21 is a diagram illustrating a configuration of a short circuit prevention portion 100 according to the second embodiment. Specifically, FIG. 21 is a cross-sectional view corresponding to FIG. 6. In FIG. 21, the configuration of the +Z-axis side along the central axis Ax of the ultrasonic vibrator 53 from the distal end side Ar1 of the first electrode 5410 is illustrated, and other features of the ultrasonic vibrator 53 are omitted

Short circuit preventing portion 100 according to the second embodiment is a part of the second electrode 5420 as shown in FIG. 21. Specifically, according to the second embodiment and as shown in FIG. 21, the inner diameter dimension of the second electrode 5420 is smaller than the inner diameter dimension of the positive electrode plate 5411, the piezoelectric element 543, the insulating plate 544, the third electrode 545, and the distal end Ar1 of the back mass 56. That is, the inner peripheral edge of the second electrode 5420 protrudes further toward the center axis Ax than the inner peripheral edge of the positive electrode plate 5411, the piezoelectric element 543, the insulating plate 544, the third electrode 545, and the end portion of the distal end side Ar1 of the back mass 56. Then, the protruding portion of the inner peripheral edge of the second electrode 5420 becomes a short circuit preventing portion 100.

Even when the short circuit preventing portion 100 according to the second embodiment described above is employed, the same effect as in the first embodiment described above is achieved.

### Modified Example 2-1

FIG. 22 is a diagram illustrating modification 2-1 of the second embodiment. Specifically, FIG. 22 is a cross-sectional view corresponding to FIG. 21. According to the present modification 2-1 shown in FIG. 22, instead of the short circuit preventing portion 100 as described in the second embodiment, the short circuit preventing portion 100 of modification 2-1 shown in FIG. 22 may be adopted. Short-circuit preventing portion 100 according to the present modification 2-1 is a part of the insulating plate 544.

Specifically, according to modification 2-1, the inner diameter dimension of the insulating plate 544 is smaller than the inner diameter dimension of the positive electrode plate 5411, the piezoelectric element 543, the second electrode 5420, the third electrode 545, and the distal end Ar1 of the back mass 56. That is, the inner peripheral edge of the insulating plate 544 protrudes further toward the center axis Ax than the inner peripheral edge of the positive electrode plate 5411, the piezoelectric element 543, the second electrode 5420, the third electrode 545, and the end portion of the distal end side Ar1 of the back mass 56. Then, the inner peripheral edge of the insulating plate 544 becomes a short-circuit preventing portion 100.

Even when a part of the insulating plate 544 is set as the short circuit preventing portion 100 as in modification 2-1 described above, similarly to the second embodiment described above it is possible to more reliably prevent a short circuit between the first electrode 5410 and the third electrode 545.

### Modified Example 2-2

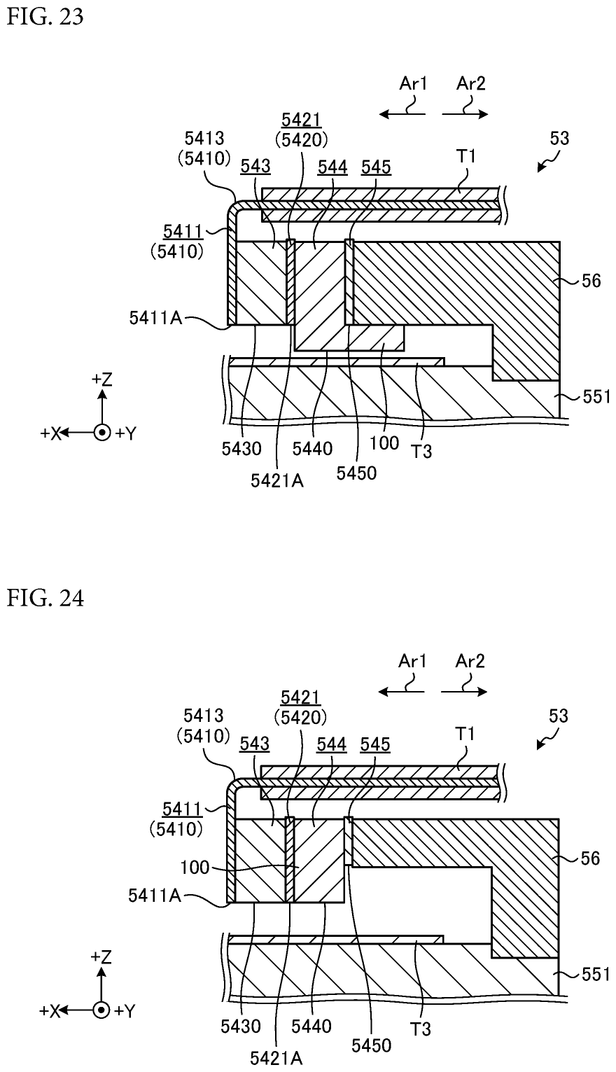

FIG. 23 is a diagram illustrating modification 2-2 of the second embodiment. Specifically, FIG. 23 is a cross-sectional view corresponding to FIG. 21. Modification 2-2 is similar to modification 2-1, but as shown in FIG. 23,

the structure of the short-circuit preventing portion 100 in modification 2-1 includes a structure on the insulating plate 544 where an inner edge portion of the insulating plate 544 is bent substantially 90° and extends along the central axis Ax direction toward the proximal end side Ar2, which creates an L-shaped cross section for the insulating plate 544.

As a further modification, the short-circuit preventing portion 100 according to modification 2-2 may be configured to extend the entire circumference in the circumferential direction around the central axis Ax, or may be configured to be provided only in a portion of the position in the circumferential direction. According to modification 2-2, since the minimum distance between conductive parts along the surface of the insulator can be sufficiently secured with respect to the 2-1 modification described above, it is possible to further and reliably prevent the short circuit between the first electrode 5410 and the third electrode 545.

### Modified Example 2-3

FIG. 24 is a diagram illustrating modification 2-3 of the second embodiment. Specifically, FIG. 24 is a cross-sectional view corresponding to FIG. 21. According to the present modification 2-3 shown in FIG. 24, instead of the short circuit preventing portion 100 as described in the second embodiment, the short circuit preventing portion 100 of modification 2-3 shown in FIG. 24 may be adopted. Short-circuit preventing portion 100 according to the present modification 2-3 is a part of the insulating plate 544.

Specifically, according to modification 2-3, the inner diameter dimension of the positive electrode plate 5411, the piezoelectric element 543, the second electrode 5420, and the insulating plate 544 is smaller than the inner diameter dimension of third electrode 545 and the end portion of the distal end side Ar1 of the back mass 56. Then, a portion of the insulating plate 544 protruding further toward the central axis Ax than the inner peripheral edge of the third electrode 545 is a short-circuit preventing portion 100.

Even when a portion of the insulating plate 544 is set as the short-circuit preventing portion 100 as in modification 2-3 described above, similarly to the second embodiment described above, it is possible to more reliably prevent a short circuit between the first electrode 5410 and the third electrode 545.

### Modified Example 2-4

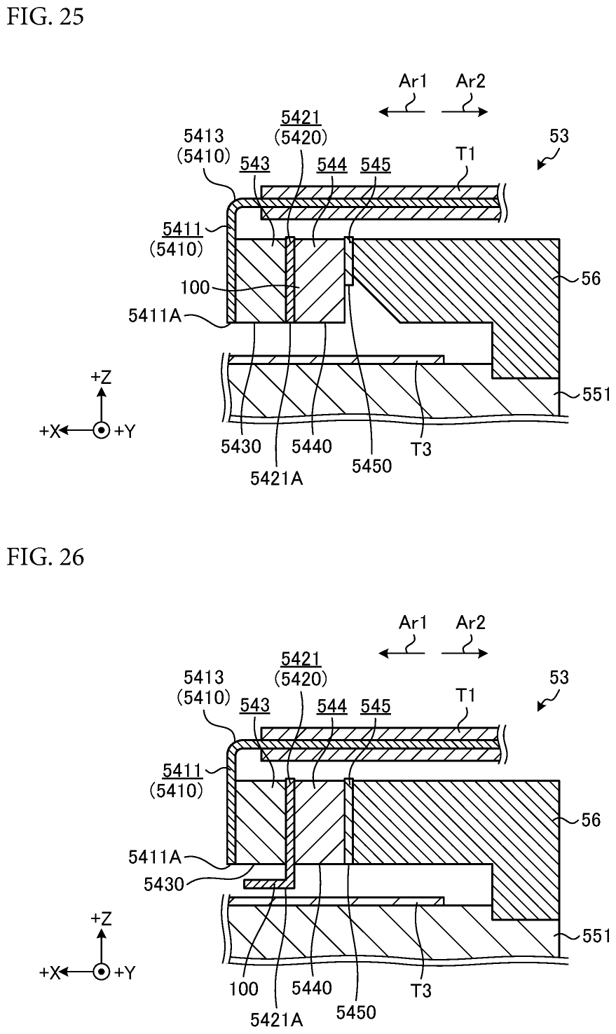

FIG. 25 is a diagram illustrating modification 2-4 of the second embodiment. Specifically, FIG. 25 is a cross-sectional view corresponding to FIG. 21.

Although the short circuit preventing portion 100 as described in modification 2-3 had the inner diameter dimension of the end portion of the distal end side Ar1 of the back mass 56 the same as the inner diameter dimension of the third electrode 545, the inner diameter dimension of the end portion of the distal end side Ar1 of the back mass 56 is not limited thereto. Thus, according to the present modification 2-4, the inner diameter of the end portion of the distal end side Ar1 of the back mass 56 may be larger than the inner diameter dimension of the third electrode 545. In a specific embodiment according to modification 2-4 and as shown in FIG. 25, the inner diameter dimension of the end portion of the distal end side Ar1 of the back mass 56 can be the same as the inner diameter of the positive electrode plate 5411, the piezoelectric element 543, the second electrode 5420, and the insulating plate 544 and the end portion of the back mass 56 may further have a chamfered corner portion at the tip of the inner peripheral edge on the distal end side Ar1.

### Modified Example 2-5

FIG. 26 is a diagram illustrating modification 2-5 of the second embodiment. Specifically, FIG. 26 is a cross-sectional view corresponding to FIG. 21.

Modification 2-5 is similar to the second embodiment, but as shown in FIG. 26, in addition to the portion protruding inwardly along the radial direction of the second electrode 5420 from a portion of an inner edge of the second electrode 542, the short-circuit prevention portion 100 also includes a portion of the second electrode 5420 extended in a bent state substantially 90° that extends toward the distal end side Ar1 along the inner edge portion of the piezoelectric element 543 and forming a structure having an L-shaped cross section. The portion in a bent state extends toward the distal end side Ar1 to cover at least a portion of the inner peripheral edge of the piezoelectric element 543, preferably to cover the entire inner peripheral edge of the piezoelectric element 543.

According to modification 2-5, since the structure is provided in which the short circuit between the first electrode 5410 and the second electrode 5420 is positively performed, it is possible to further reliably prevent the short circuit between the first electrode 5410 and the third electrode 545.

### Modified Example 2-6

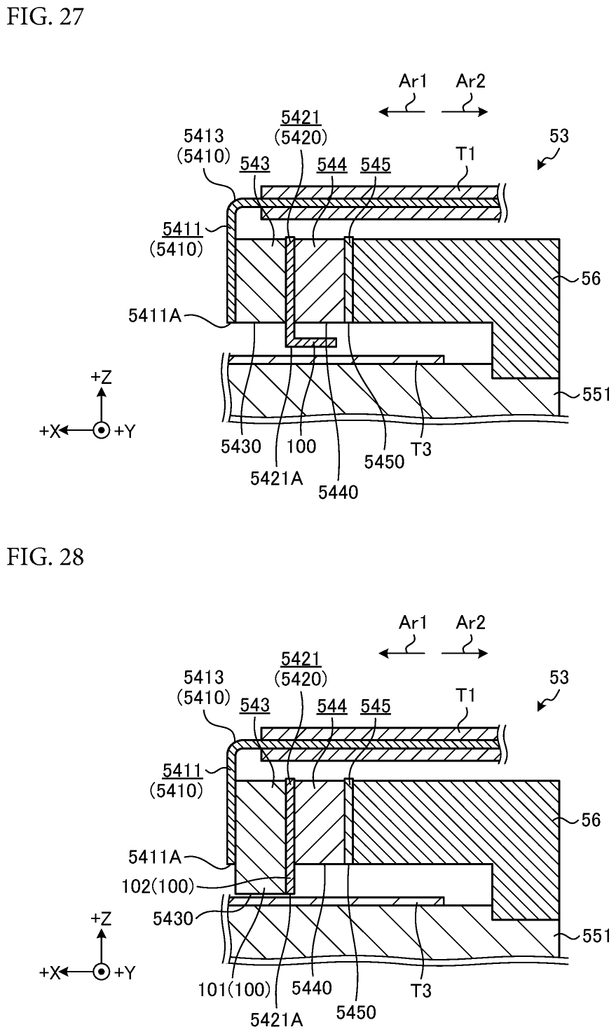

FIG. 27 is a diagram illustrating modification 2-6 of the second embodiment. Specifically, FIG. 27 is a cross-sectional view corresponding to FIG. 21.

Modification 2-6 is similar to modification 2-5, but as shown in FIG. 27, the portion of the second electrode 5420 extended in a bends substantially 90° to a bent state and extends toward the proximal end side Ar2 along the inner edge portion of insulating plate 544 and forming a structure having an L-shaped cross section. The portion in a bent state extends toward the proximal end side Ar2 to cover at least a portion of the inner peripheral edge of the insulating plate 544 and to cover at least a portion of the entire circumference.

According to modification 2-6, since the structure is provided in which the short circuit between the first electrode 5410 and the second electrode 5420 is positively performed, it is possible to further reliably prevent the short circuit between the first electrode 5410 and the third electrode 545.

### Modified Example 2-7

FIG. 28 is a diagram illustrating modification 2-7 of the second embodiment. Specifically, FIG. 28 is a cross-sectional view corresponding to FIG. 21.

According to the present modification 2-7 shown in FIG. 28, instead of the short circuit preventing portion 100 as described in the second embodiment, the short circuit preventing portion 100 of modification 2-7 shown in FIG. 28 may be adopted. Short-circuit preventing portion 100 according to the present modification 2-7 includes a first short-circuit preventing portion 101, which is a part of the piezoelectric element 543, and a second short-circuit preventing portion 102, which is a part of the second electrode 5420.

Specifically, in modification 2-7, the inner diameter dimension of the piezoelectric element 543 and the second electrode 5420 is set to be smaller than the inner diameter dimension of the positive electrode plate 5411, the insulating plate 544, the third electrode 545, and the end portion of the distal end side Ar1 of the back mass 56. That is, the inner peripheral edge of the piezoelectric element 543 and the second electrode 5420 extends further toward the center axis Ax than the inner peripheral edge of the positive electrode plate 5411, the insulating plate 544, the third electrode 545, and the end portion of the distal end side Ar1 in the back mass 56. Then, the extending portion of the inner peripheral edge of the piezoelectric element 543 becomes a first short-circuit preventing portion 101. Further, the extending portion of the inner peripheral edge of the second electrode 5420 becomes a second short-circuit prevention portion 102.

According to modification 2-7, in addition to a part of the second electrode 5420, since a part of the piezoelectric element 543 is the short-circuit preventing portion 100, a short circuit between the first electrode 5410 and the third electrode 545 can be more reliably prevented. Since a part of the piezoelectric element 543 is the short-circuit preventing portion 100, the second electrode 5420 may have the same inner diameter as the first electrode 5410 or the third electrode 545.

### Modified Example 2-8

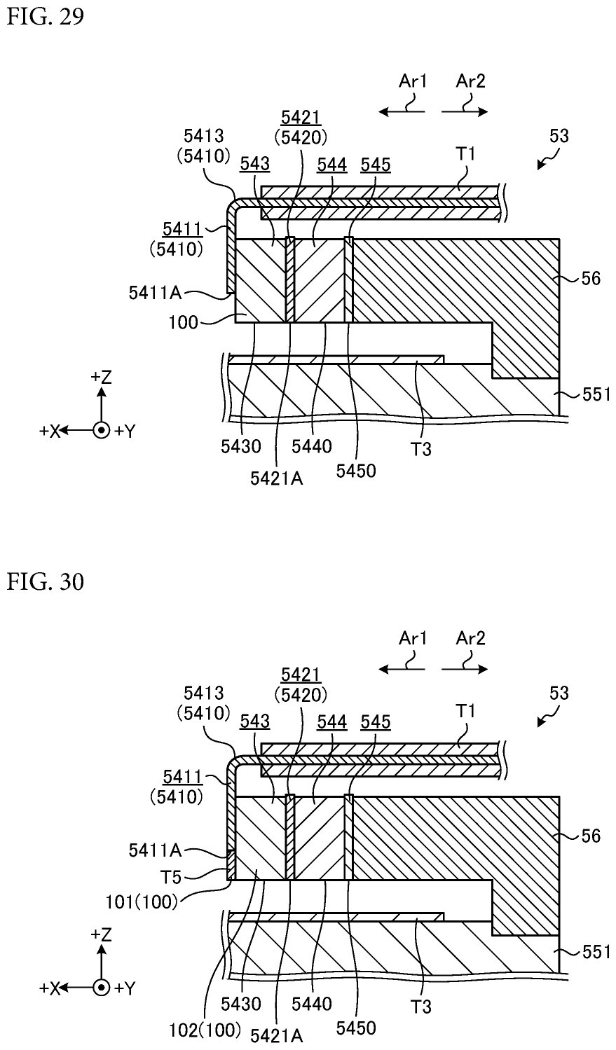

FIG. 29 is a diagram illustrating modification 2-8 of the second embodiment. Specifically, FIG. 29 is a cross-sectional view corresponding to FIG. 21.

According to the present modification 2-8 shown in FIG. 29, instead of the short-circuit preventing portion 100 described in the second embodiment described above, it may be adopted a short-circuit preventing portion 100 according to the present modification 2-8 shown in FIG. 29. Short-circuit preventing portion 100 according to the present modification 2-8 includes a part of the piezoelectric element 543.

Specifically, in modification 2-8 and as shown in FIG. 29, the inner diameter dimension of the positive electrode plate 5411 constituting the first electrode 5410 is smaller than the inner diameter dimension of the piezoelectric element 543, the second electrode 5420, the insulating plate 544, the third electrode 545, and the end portion of the distal end side Ar1 of the back mass 56. Then, a portion of the piezoelectric element 543 protruding toward the central axis Ax further than the inner peripheral edge of the positive electrode plate 5411 becomes a short-circuit preventing portion 100.

Even when employing a structure that reduces the inner diameter dimension of the positive electrode plate 5411 constituting the first electrode 5410 as in the present modification 2-8 described above, it is possible to increase the minimum distance between conductive parts along the surface of the insulator and, similarly to the second embodiment, a short circuit between the first electrode 5410 and the third electrode 545 can be more reliably prevented.

### Modified Example 2-9

FIG. 30 is a diagram illustrating modification 2-9 of the second embodiment. Specifically, FIG. 30 is a cross-sectional view corresponding to FIG. 21.

In modification 2-9 described above, an annular insulating shim T5 having an electrical insulating property may be disposed on the inner peripheral edge of the positive electrode plate 5411 constituting the first electrode 5410. The size of the outer diameter of the insulation shim T5 is substantially the same size as the inner diameter of the positive electrode plate 5411. The inner diameter of the insulation shim T5 is the same as the inner diameter of the piezoelectric element 543. Then, the short-circuit preventing portion 100 includes a first short-circuit preventing portion 101, which is insulating shim T5, and a second short-circuit preventing portion 102, which is a part of the piezoelectric element 543.

According to modification 2-9, in addition to a part of the piezoelectric element 543, since the insulation shim T5 is also a short-circuit preventing portion 100, a short circuit between the first electrode 5410 and the third electrode 545 can be more reliably prevented.

### Modified Example 2-10

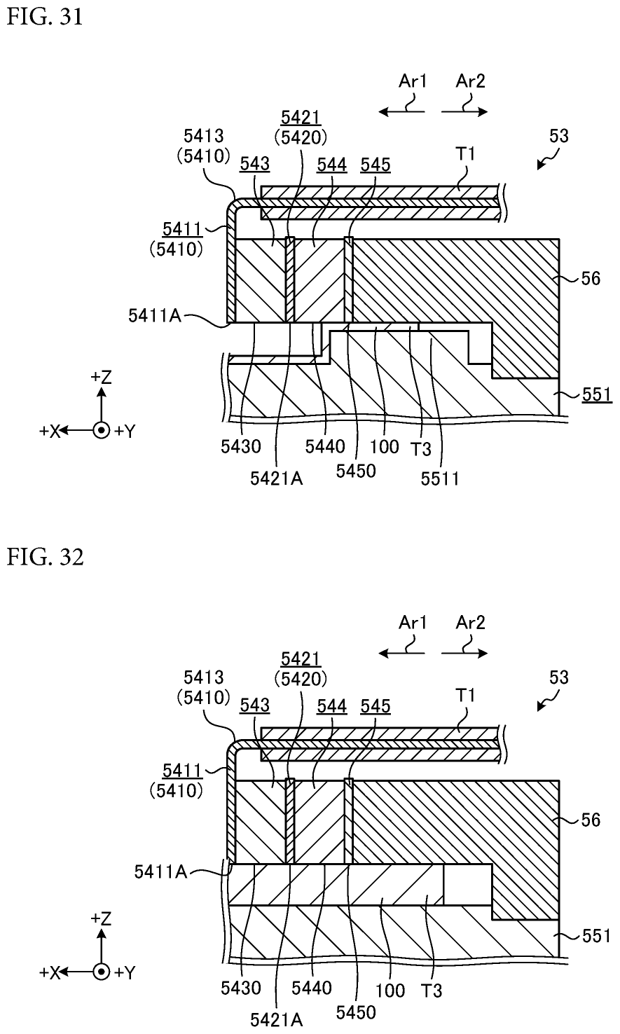

FIG. 31 is a diagram illustrating modification 2-10 of the second embodiment. Specifically, FIG. 31 is a cross-sectional view corresponding to FIG. 21.

According to the present modification 2-10 shown in FIG. 31, instead of the short-circuit preventing portion 100 described in the second embodiment described above, it may be adopted a short-circuit preventing portion 100 according to the present modification 2-10 shown in FIG. 31. Short-circuit preventing portion 100 according to the present modification 2-10 includes a part of the insulating tube T3.

Specifically, according to the present modification 2-10 and as shown in FIG. 31, the outer peripheral surface of the first mounting portion 551 includes an annular flange portion 5511 that bulges outward. Annular flange portion 5511 is at a position facing the inner peripheral edge of the third electrode 545. Further, the insulating tube T3 covers at least a portion of the outer peripheral surface of the first mounting portion 551 including at least a portion of the flange portion 5511. As the insulating tube T3 is positioned to cover the outer peripheral surface of the flange portion 5511 opposing the entire inner periphery end of the third electrode 545, a short-circuit preventing portion 100 is created.

Even when a part of the insulating tube T3 is set as the short circuit preventing portion 100 as in modification 2-10 described above, it is possible to more reliably prevent a short circuit between first electrode 5410 and the third electrode 545 in the same manner as in the second embodiment described above.

### Modified Example 2-11

FIG. 32 is a diagram illustrating modification 2-11 of the second embodiment. Specifically, FIG. 32 is a cross-sectional view corresponding to FIG. 21.

According to the present modification 2-11 shown in FIG. 32, instead of the short-circuit preventing portion 100 described in the second embodiment described above, it may be adopted a short-circuit preventing portion 100 according to the present modification 2-11 shown in FIG. 32. Short-circuit preventing portion 100 according to the present modification 2-11 includes a part of the insulating tube T3.

Specifically, according to the present modification 2-11 and as shown in FIG. 32, the insulating tube T3 has a thickness dimension that is larger than the insulating tube T3 described in modification 2.10. Consequently, the insulating tube T3 of modification 2-11 contacts each of the positive electrode plate 5411, the piezoelectric element 543, the second electrode 5420, the insulating plate 544, the third electrode 545, and the inner peripheral edge of the end portion of the distal end side Ar1 of the back mass 56. Then, the insulating tube T3 in contact with inner peripheral edge of the positive electrode plate 5411, the piezoelectric element 543, the second electrode 5420, the insulating plate 544, the third electrode 545, and the end portion of the distal end side Ar1 of the back mass 56 is a short-circuit preventing portion 100.

Even when a part of the insulating tube T3 is set as the short circuit preventing portion 100 as in modification 2-11 described above, it is possible to more reliably prevent a short circuit between the first electrode 5410 and the third electrode 545 in the same manner as in the second embodiment described above.

### Modified Example 2-12

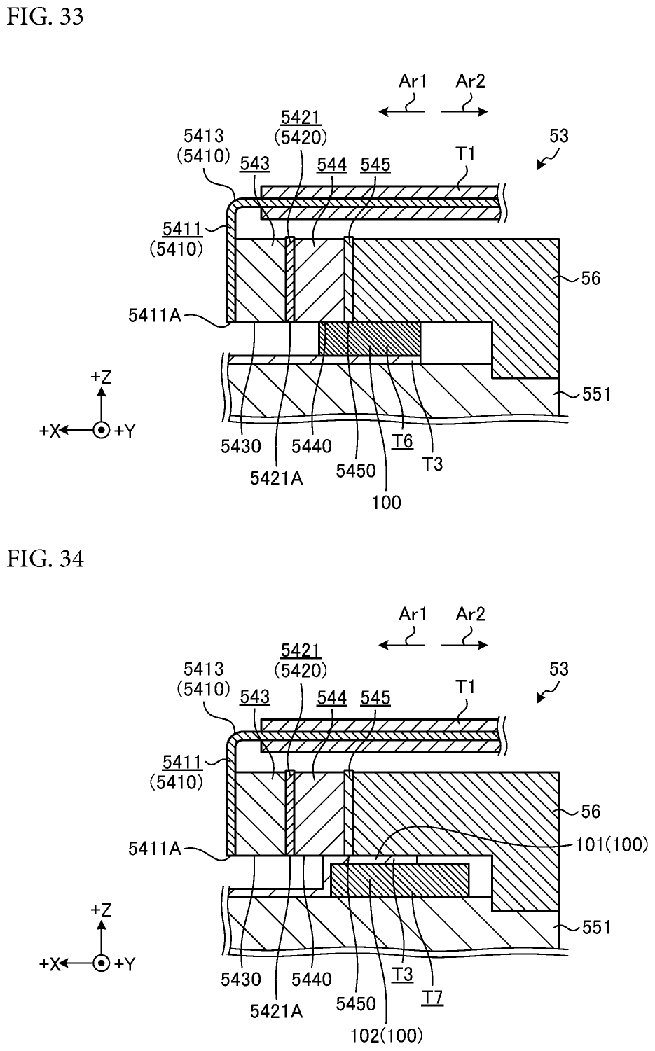

FIG. 33 is a diagram illustrating modification 2-12 of the second embodiment. Specifically, FIG. 33 is a cross-sectional view corresponding to FIG. 21.

According to the present modification 2-12 shown in FIG. 33, instead of the short-circuit preventing portion 100 described in the second embodiment described above, it may be adopted a short-circuit preventing portion 100 according to the present modification 2-12 shown in FIG. 33. Short-circuit preventing portion 100 according to the present modification 2-12 includes a part of the insulating tube T6.

Specifically, as shown in FIG. 33, the insulation tube T6 is composed of a material having an electrically insulating property and is disposed to cover the outer peripheral surface of the end portion of the proximal end side Ar2 of insulating tube T3. Then, the insulating tube T6 in contact with the inner peripheral edge of the third electrode 545 is a short-circuit preventing portion 100.

Even when the insulating tube T6 is set as the short-circuit preventing portion 100 as in modification 2-12 described above, similarly to the second embodiment, it is possible to more reliably prevent a short circuit between the first electrode 5410 and the third electrode 545.

### Modified Example 2-13

FIG. 34 is a diagram illustrating modification 2-13 of the second embodiment. Specifically, FIG. 34 is a cross-sectional view corresponding to FIG. 21.

According to the present modification 2-13 shown in FIG. 34, instead of the short-circuit preventing portion 100 described in the second embodiment described above, it may be adopted a short-circuit preventing portion 100 according to the present modification 2-13 shown in FIG. 34. Short-circuit preventing portion 100 according to the present modification 2-13 includes a first short-circuit preventing portion 101, which is a part of the insulating tube T3, and a second short-circuit preventing portion 102, which is an insulating collar T7.

Specifically, the insulating collar T7 is composed of a material having an electrical insulation property and has an annular shape. As shown in FIG. 34, the first mounting portion 551 is inserted into the insulating collar T7 so that the insulating collar is located on the outer peripheral surface of the first mounting portion 551 at a position that is fixed facing the inner peripheral edge of the third electrode 545. Further, the insulating tube T3 is disposed to cover the outer peripheral surface of the first mounting portion 551 including the insulating collar T7. Then, the portion of the insulating tube T3 covering the outer peripheral surface of the insulating collar T7 and disposed abutting the inner peripheral edge of the third electrode 545 is a first short-circuit preventing portion 101. Further, the insulating collar T7 is a second short-circuit preventing portion 102.

According to modification 2-13, in addition to the insulating tube T3, since the insulating collar T7 is also a short-circuit preventing portion 100, a short circuit between the first electrode 5410 and the third electrode 545 can be more reliably prevented.

### Other Embodiments

While embodiments for carrying out the present invention have been described so far, the present invention is not to be limited only by the embodiments described above.

In the first and second embodiments and modified embodiments 1-1 to 1-13 and 2-1 to 2-13 described above, the ultrasonic treatment instrument according to the present invention is configured to impart both ultrasonic energy and high frequency energy to the target site, but is not limited thereto. The ultrasonic treatment tool according to the present invention may be configured to impart only ultrasonic energy to a target site, and may be configured to impart the ultrasonic energy and at least one of high frequency energy and thermal energy. Here, “imparting heat energy to a target site” means transmitting heat generated in a heater or the like to a target site.

### DESCRIPTION OF SYMBOLS

1 Treatment system
2 Ultrasonic treatment device
3 Control system
4 Handpiece
5 Ultrasonic transducer
6 Housing
7 Movable handle
8 Switch
9 Rotary knob
10 Shaft
10A pins
10B notch
11 Opening and closing mechanism
12 Jaw
13 Vibration transmission member
14 Terminal unit
15 Terminal holding portion
16 Handpiece side terminal
17 End effector
51 TD case
52 TD side terminal
53 Ultrasonic vibrator
54 Transducer body
55 Front mass
56 Back mass
61 Case body
62 Fixed handle
71 Handle base
72 Operation unit
73 Connecting part
100 Short Circuit Prevention portion
101 First short-circuit prevention portion
102 Second short-circuit prevention portion
103 Third short-circuit prevention portion
111 Inner pipe
112 Holding portion
113 Slider receiver
114 Slider
115 Biasing member
121 Second Pin
131 Treatment portion
161 HF return electrode terminal
162 HF active electrode terminal
163 U.S. return electrode terminal
164 U.S. active electrode terminal
511 TD case body
512 Terminal holding portion
521 HF active electrode terminal
522 U.S. return electrode terminal
523 U.S. active electrode terminal
541 First electrode unit
542 Second electrode unit
543 Piezoelectric material or element
544 Insulating plate
545 Third electrode
546 Insulating plate
551 First mounting portion
552 Cross-sectional area change portion
553 Second mounting portion
1111 Arm portion
1121 HF return electrode terminal
1122 Electrical path
5410 First electrode
5411 Positive electrode plate
5411A opening
5412 Positive electrode wiring part
5413 Positive electrode terminal
5420 Second electrode
5421 Negative electrode plate
5421A opening
5422 Negative electrode wiring part
5423 Negative electrode terminal
5430 Opening
5431 First end surface
5432 Second end surface
5440,5450 Opening
5511 Flange portion
Ar1 distal end side
Ar2 proximal end side
Ax central axis
C Electrical cable
L1 Straight line
Rx1 first rotational axis
Rx2 second rotational axis
T1˜T4,T6 insulating tube
T5 Insulating shim
T7 Insulating collar
TI inner tube
TO Outer tube

What is claimed is:

1. An energy device, comprising:
a piezoelectric material having a cylindrical shape, the piezoelectric material including a first surface and a second surface opposing to the first surface, the piezoelectric material configured to generate an ultrasonic vibration for treating living tissue by an ultrasonic energy;
a first electrode contacting the first surface of the piezoelectric material, the first electrode configured to apply an ultrasonic driving voltage to the piezoelectric material for generating the ultrasonic vibration;
a second electrode contacting the second surface of the piezoelectric material, the second electrode configured to apply a reference voltage to the piezoelectric material for generating the ultrasonic vibration;
an electrical insulated plate having an electrical insulating property, the electrical insulated plate opposing to the second surface of the piezoelectric material with the second electrode interposed between the electrical insulated plate and the piezoelectric material, the electrical insulated plate contacting the second electrode;
a third electrode opposing to the second electrode with the electrical insulated plate interposed between the third electrode and the second electrode, the third electrode configured to apply a high-frequency electric power for treating living tissue by a high-frequency energy; and
a short-circuit prevention portion configured to prevent a short-circuit between the first electrode and the third electrode.

2. The energy device according to claim 1, wherein the short-circuit prevention portion is configured to prevent the short-circuit between the first electrode and the third electrode from occurring on an outer peripheral surface side of the piezoelectric material.

3. The energy device according to claim 1, wherein the short-circuit prevention portion is configured to prevent the short-circuit between the first electrode and the third electrode from occurring on an inner peripheral surface side of the piezoelectric material.

4. The energy device according to claim 1, wherein the short-circuit prevention portion is the second electrode and a portion of the second electrode intersects an imaginary straight line, where the imaginary straight line is an imaginary straight line without a circumferential component between an outermost side of the third electrode and the first electrode.

5. The energy device according to claim 4, wherein the imaginary straight line without circumferential component is the shortest imaginary straight line without circumferential component between the outermost side of the third electrode and any location on the first electrode not covered by an insulating tube.

6. The energy device according to claim 4, wherein the imaginary straight line without circumferential component is one of: (i) the shortest imaginary straight line without circumferential component having a first end at any location within a bend in the first electrode where a positive electrode plate portion of the first electrode transitions to become a positive electrode terminal portion of the first electrode and a second end at the outermost side of the third electrode; (ii) the shortest imaginary straight line without circumferential component between the outermost side of the third electrode and any location on a positive electrode plate portion of the first electrode not covered by an insulating tube, and (iii) the shortest imaginary straight line without circumferential component between the outermost side of the third electrode and any location on a positive electrode terminal portion of the first electrode not covered by an insulating tube.

7. The energy device according to claim 4, wherein the portion of the second electrode extends in a radial direction beyond the third electrode.

8. The energy device according to claim 7, wherein the first electrode has a protrusion that extends in the radial direction, and
wherein at least a portion of the short-circuit prevention portion is located at a position in a circumferential direction around a central axis of the piezoelectric material that is the same as a position of the protrusion of the first electrode.

9. The energy device according to claim 7, wherein the short-circuit prevention portion has a protruded part that is bent to extend along a central axis of the piezoelectric material.

10. The energy device according to claim 1, wherein the short-circuit prevention portion is a structure including an electrical insulated material.

11. The energy device according to claim 10, wherein the first electrode has a protrusion that extends in a radial direction of the piezoelectric material, and
wherein a portion of the short-circuit prevention portion is located at position in a circumferential direction around a central axis of the piezoelectric material that is different than a position of the protrusion of the first electrode.

12. The energy device according to claim 10, wherein the short-circuit prevention portion is a portion of the electrical insulated plate and the portion of the electrical insulated plate extends in a radial direction beyond the third electrode.

13. The energy device according to claim 10, wherein the short-circuit prevention portion is an electrical insulated tube disposed to cover an outer peripheral surface of the third electrode.

14. The energy device according to claim 10, wherein the short-circuit prevention portion is an insulated tube disposed to cover an inner peripheral surface of the third electrode.

15. A treatment instrument, comprising:
an end effector configured to apply an ultrasonic energy and a high-frequency energy to living tissue for treating living tissue; and
an ultrasonic transducer configured to generate the ultrasonic vibration for treating living tissue by an ultrasonic energy, the ultrasonic transducer comprising:
a piezoelectric material having a cylindrical shape, the piezoelectric material including a first surface and a second surface opposing to the first surface, the piezoelectric material configured to generate an ultrasonic vibration for treating living tissue by the ultrasonic energy,
a first electrode contacting the first surface of the piezoelectric material, the first electrode configured to apply an ultrasonic driving voltage to the piezoelectric material for generating the ultrasonic vibration,
a second electrode contacting the second surface of the piezoelectric material, the second electrode configured to apply a reference voltage to the piezoelectric material for generating the ultrasonic vibration,
an electrical insulated plate having an electrical insulating property, the electrical insulated plate opposing to the second surface of the piezoelectric material with the second electrode interposed between the electrical insulated plate and the piezoelectric material, the electrical insulated plate contacting the second electrode,
a third electrode opposing to the second electrode with the electrical insulated plate interposed between the third electrode and the second electrode, the third electrode configured to apply a high-frequency electric power for treating living tissue by a high-frequency energy, and
a short-circuit prevention portion configured to prevent a short-circuit between the first electrode and the third electrode.

16. The treatment instrument according to claim 15, further comprising,
a power transmission member configured to transmit the ultrasonic vibration generated by the ultrasonic transducer from a proximal part to a distal part,
wherein the end effector is a part of a distal side of the power transmission member,
wherein the ultrasonic transducer includes:
a front mass connected mechanically to a proximal side of the power transmission member, and
a back mass electrically connecting to the front mass, the back mass attaching the piezoelectric material, the first electrode, the second electrode, the insulation plate, and the third electrode to the ultrasonic transducer,
wherein the end effector is configured to be supplied with the high-frequency electric power via the third electrode, the back mass, the front mass, and the power transmission member.

17. The treatment instrument according to claim 15, wherein the short-circuit prevention portion is configured to prevent the short-circuit between the first electrode and the third electrode from occurring on an outer peripheral surface side of the piezoelectric material.

18. The treatment instrument according to claim 15, wherein the short-circuit prevention portion is configured to prevent the short-circuit between the first electrode and the third electrode from occurring on an inner peripheral surface side of the piezoelectric material.

19. The treatment instrument according to claim 15, wherein the short-circuit prevention portion is the second electrode and a portion of the second electrode intersects an imaginary straight line, where the imaginary straight line is an imaginary straight line between the third electrode and the first electrode having an axial component of the piezoelectric material and without a circumferential component.

20. The treatment instrument according to claim 19, wherein the portion of the second electrode extends in a radial direction beyond the third electrode.

21. The treatment instrument according to claim 15, wherein the short-circuit prevention portion is a structure including an electrical insulated material.
